(19) United States
(12) Patent Application Publication
(10) Pub. No.: US 2005/0201936 A1
(43) Pub. Date: Sep. 15, 2005

(54) Models for viral-based cancer therapy

(76) Inventors: William S. M. Wold, Chesterfield, MO (US); Karoly Toth, St. Louis, MO (US); Maria A. Thomas, Crestwood, MO (US)

Correspondence Address: FULBRIGHT & JAWORSKI L.L.P., 600 CONGRESS AVE., SUITE 2400, AUSTIN, TX, 78701, US

(21) Appl. No.: 11/057,710

(22) Filed: Feb. 14, 2005

Related U.S. Application Data
(60) Provisional application No. 60/544,106, filed on Feb. 12, 2004.

(51) Int. Cl. C12N 15/861; A61K 49/00; C12Q 1/70

(52) U.S. Cl. 424/9.2; 435/456; 435/5

(57) ABSTRACT

The present invention relates to the use of cotton rats and Syrian hamsters as models for adenoviral gene therapy. In particular, the models are directed to the examination of the ability of various replicative, non-replicative and conditionally-replicative adenovirus vectors to treat cancer.

### BACKGROUND OF THE INVENTION

The present invention claims benefit of priority to U.S. Provisional Ser. No. 60/544,106, filed Feb. 12, 2004, the entire contents of which are hereby incorporated by reference.

1. Field of the Invention

The invention relates to the fields of veterinary science, virology, oncology and pathology. More particularly, the invention relates to the use of cotton rats and Syrian hamsters as models for adenoviral therapies, such as cancer therapies.

2. Description of Related Art

Adenoviruses (Ad) infect humans but are usually mild pathogens that can cause respiratory illness or conjunctivitis. Under laboratory conditions, some human strains can transform cells in culture, but the viruses are not considered oncogenic. As a result of their human tropism and lack of severe disease, adenoviruses have been the subject of intense examination from the standpoint of gene therapy. Numerous clinical trials are ongoing using Ad vectors to deliver a variety of therapeutic genes.

The standard model for examining the anti-tumor efficacy of replication competent Ad vectors is the immunodeficient mouse/human tumor xenograft model. The use of this chimeric system is required because human adenoviruses are very species-specific and do not replicate well in most rodent cell lines or tissues. The further need for immunodeficient mice, to avoid rejection of the human tumor cells, complicates matters considerably.

For example, although most oncolytic vectors are designed not to replicate in non-cancerous cells, thus far the only way to test this hypothesis was to conduct tissue culture experiments and human clinical studies. In addition, the host immune system can affect the outcome of the treatment—it can eliminate the virus before it could assert any effect on the tumor, or, conversely it could accentuate the anti-tumor effect of the vector by mounting an immune response against the tumor itself. Again, this effect can only be investigated in humans.

Thus, there remains a need for an improved animal for testing of therapeutic adenoviral vectors, particularly those that are replicative.

### SUMMARY OF THE INVENTION

Thus, in accordance with the present invention, there is provided a method of evaluating an adenoviral vector for anti-tumor effects comprising (a) providing a Syrian hamster that comprises a hamster cancer cell; (b) administering to said hamster a recombinantly-engineered adenoviral vector; and (c) assessing the effect of said adenoviral vector on said cancer cell. In another embodiment, there is provided a method of evaluating an adenoviral vector for anti-tumor effects comprising (a) providing a cotton rat that comprises a cotton rat cancer cell; (b) administering to said cotton rat an adenoviral vector; and (c) assessing the effect of said adenovirus on said cancer cell. The adenoviral vector may be a recombinantly-engineered adenoviral vector.

In various other embodiments, the adenoviral vector may be replication-competent or replication-deficient. It may overexpresses ADP relative to wild-type adenovirus serotype 5. The adenoviral vector may be selected from VRX-001, VRX-002, VRX-003, VRX-004, VRX-005, VRX-006, VRX-007, VRX-008, VRX-009, VRX-010, VRX-011, VRX-012, VRX-013, VRX-014, VRX-015, VRX-016, VRX-017, VRX-018, VRX-019, VRX-020, VRX-021, INGN:201, INGN:241, or INGN:251. The adenoviral vector may lack one or more regions selected from the E1, E2, E3, E4, protein pIX, protein IVₐ₂, and major late transcription units.

The adenoviral vector may comprise a heterologous coding sequence, such as for a tumor suppressor, an inducer of apoptosis, a cell cycle regulator, a toxin, an enzyme, a hormone, a cytokine, an antisense DNA or RNA directed against an oncogene, an siRNA, a single-chain antibody, an inhibitor of angiogenesis, a metalloprotease inhibitor or a peptide hormone. The tumor suppressor may be p53, mda-7, Rb, p16, or PTEN. The inducer of apoptosis may be Bax, Bad, Bik, Bid, or Bcl-Xₛ. The cell cycle regulator may be p300/CBP. The toxin may be pertussis toxin or ricin. The enzyme may be HSV-tk. The hormone may be a LHRH analog, estrogen, progestin or an anti-androgen. The antisense DNA or RNA may be directed to ras, raf, myb, myc, or src. The cytokine may be tumor necrosis factor alpha, Fas ligand, tumor necrosis factor-related apoptosis-inducing ligand (TRAIL), IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-24, interferon-α, interferon-β, and interferon-γ. The siRNA may be directed to ras, raf, myb, myc, or src. The single-chain antibody may be directed to ras, raf, myb, myc, or src.

The cancer cell may be comprised within a solid tumor or within a metastatic lesion. The cancer cell may be hamster cancer cell such as a pancreatic carcinoma cell, a leiomyosarcoma cell, or a kidney tumor-forming cell. The cotton rat cancer cell may be a sarcoma cell. Administering may comprise intratumoral or intralesional injection, local, regional or systemic administration, subcutaneous, intranasal, intramuscular, intravenous, intra-arterial, intraperitoneal or oral administration.

Assessing may comprise measuring a change in tumor volume or diameter following treatment with said adenoviral vector, measuring metastatic growth following treatment with said adenoviral vector, measuring development of metastases following treatment with said adenoviral vector, measuring tumor cell apoptosis following treatment with said adenoviral vector, measuring tumor necrosis following treatment with said adenoviral vector, measuring tumor infiltration of adjacent tissues following treatment with said adenoviral vector, measuring production of a tumor-derived compound following treatment with said adenoviral vector, measuring adenoviral-based toxicity in said hamster, or measuring adenoviral-based death of said hamster. The adenoviral vector may be administered more than once, and assessing may be performed once at the conclusion of all administrations, or between at least a first and a second administration, and the method may further comprise administering a second non-adenoviral therapy to said hamster, and assessing the combined effect of said adenoviral vector and said second non-adenoviral therapy.

As used herein the specification, “a” or “an” may mean one or more. As used herein in the claim(s), when used in conjunction with the word “comprising”, the words “a” or “an” may mean one or more than one. As used herein “another” may mean at least a second or more. Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Ad5 and VRX-007 establish infections and express “early” and “late” proteins in the LCRT cotton rat cell line. A549 and LCRT cells were mock infected or infected with 50 PFU/cell of Ad5 or VRX-007. The cells were harvested at 24, 36, 48, and 72 h p.i. Ten μg of protein from each sample was separated by SDS-PAGE on a 10% gel. Proteins were transferred to Immobilon-P membrane and were then probed with antibodies to the Ad5 proteins E1A (immediate early), DBP (early and late), Fiber (late), ADP (primarily late), or pVIII (late).

FIG. 2—Ads replicate in the LCRT cotton rat cell line. LCRT cells were infected with 50 PFU/cell of Ad5 or VRX-007. After infection, the cells were washed three times with medium, and then harvested together with medium at daily intervals from 0 to 8 days post infection. The harvested samples were titered on A549 human lung adenocarcinoma cells, and the data were plotted as viral titer vs. time post infection.

FIGS. 3A and 3B—VRX-007 and Ad5 show extensive spread of viral infection on the LCRT cotton rat cell line at 5 days post-infection. Cells were infected with VRX-007 or Ad5 at 1×10⁻² PFU/cell. At 5 days post-infection, cells were fixed and then immunostained for E1A and DNA binding protein (DBP) or for DBP and Fiber. Left and right panels of each pair show the same field. Uniform DBP patterns indicate early stages of infection, while punctate patterns are seen in late infection. Fiber staining is an indication of late infection.

FIGS. 4A and 4B—VRX-007 suppresses the growth of LCRT tumors in cotton rats. Subcutaneous cotton rat LCRT tumors were generated by injecting 2×10⁶ cells into both flanks of cotton rats. When tumors reached an average volume of 200 μl (VRX-007 group) or 300 μl (mock group), they were injected with buffer or 8.5×10¹⁰ particles of VRX-007 per injection. Injections were given on day 0, 1, 2, 3, 4, 7, 8, 9, 10, and 11. (FIG. 4A) The mean tumor volume is plotted vs. time. (FIG. 4B) Data was further analyzed using SPSS© software. The median (black horizontal bar within grey box), interquartile range (grey box), and standard deviation of the tumor volumes of each group on day 13 are shown.

FIG. 5—Intratumoral injection of VRX-007 delays the development of subcutaneous LCRT tumor transplants in cotton rats. Cotton rats bearing LCRT tumors were injected with buffer or a total dose of 3.4×10¹¹ VP of intact or UV-inactivated VRX-007. The tumors were measured on the indicated days after injection of cells. The circles denote outliers (volumes between 1.5 and 3 box lengths from edge of box). The asterisks are extreme outliers (volumes more than 3 box lengths from box edge). The significance was tested by one-way ANOVA and Student's t-Test (VRX-007 vs. mock or UV-inactivated p<0.001).

FIG. 6—LCRT cells pre-infected with VRX-007 show reduced tumor growth. LCRT cells in culture dishes were infected with 100 PFU/cell of VRX-007 then trypsinized. Infected cells were injected into cotton rats (100% infected) or mixed with uninfected LCRT cells at a ratio of 1:5 (20% infected). As controls, uninfected LCRT (mock) cells or LCRT cells that had been killed by freezing and thawing (dead mock) were also injected into cotton rats. Each flank was injected with 100 μl of 4×10⁶ LCRT cells of one of the samples. Tumor volume was measured periodically. Data were further analyzed using SPSS software. The median, interquartile range, and standard deviation of tumor volumes at twelve days post injection are shown.

FIG. 7—VRX-007 infects and progresses into the late phase in Syrian hamster tumor cell lines. Syrian hamster cell lines infected at 50 PFU/cell with VRX-007 were fixed and stained at 24 or 48 h p.i. At 24 h p.i., cells were immunostained for E1A and DBP. At 48 h p.i., cells were immunostained for Fiber and DBP. At both times, nuclear DNA was stained with DAPI. The antisera used did not stain mock-infected cells.

FIG. 8—Hamster cell lines support Ad replication and entry into the late phase of infection. Hamster PC1, PC1.0, HaK, DDT, MF-2 and human A549 cells were mock infected or infected at an MOI of 100 PFU/cell with VRX-007. Cell lysate proteins were prepared at 1, 2, and 3 days p.i., separated by SDS-PAGE, and transferred to Immobilon-P membrane. The membrane was probed with a rabbit serum against Ad5 late proteins.

FIG. 9—VRX-007 replicates in three of the four Syrian hamster cell lines tested. Cells in 35 mm dishes were infected at a 100 PFU/cell to ensure a synchronous infection and dishes were harvested at 0, 2, 4, and 6 days p.i. The virus recovered from each timepoint was titered by plaque assay of the crude lysate on A549 cells and the total PFU recovered for each infection was plotted.

FIG. 10—VRX-007 is able to spread in hamster cell lines. Cells infected with VRX-007 at multiplicities ranging from 10¹ to 10⁻⁴ PFU/cell were fixed, stained for Ad proteins E1A and DBP, and scanned for areas of spread at 5 days p.i. Spread was seen to some extent in all cell lines, but the most extensive spread was seen in the PC1 cell line. No immunostaining was observed in mock-infected cells (data not shown).

FIGS. 11A and 11B—VRX-007 suppresses the growth of subcutaneous DDT1 MF-2 hamster tumors. DDT1 MF-2 cells were injected subcutaneously into both hind flanks of hamsters. Following their appearance, tumors were injected with either VRX-007 (n=10 tumors) or PBS (n=6 tumors). VRX-007 was injected for six consecutive days (3×10⁸ PFU per injection) for a total dose of 1.8×10⁹ PFU. (FIG. 11A) The mean fold increase in tumor growth is shown. Statistical analysis performed with the Student's t-test determined a statistically significant difference between the VRX-007 and buffer-injected groups, with a p value of 0.0013. (FIG. 11B) The fold increase in tumor growth for individual tumors is shown.

FIG. 12—VRX-007 suppresses the growth of subcutaneous PC1 Syrian hamster tumors. PC1 cells (1×10⁶) were injected subcutaneously into both hind flanks of hamsters. Following their appearance, tumors were injected with either VRX-007 (n=3 tumors) or PBS (n=2 tumors). VRX-007 was injected for six consecutive days (2×10¹⁰ PFU per injection) for a total dose of 1.2×10¹¹ PFU. The mean tumor volume is shown.

FIG. 13—VRX-007 treatment reduces the growth of Svrian hamster HaK cell tumors grown in Syrian hamsters. In the left panel, the median growth for each treatment group is shown, with error bars representing the standard deviation. The black arrow indicates the first day of the initial treatment schedule (days 0 through 5). The gray arrow indicates the first day of the second treatment schedule (days 16-18). Asterisks indicate statistical significance as determined by a Student's t-test. The p values as determined by the Student's t-test, for time points in the high dose group relative to the mock group that were significantly different from the buffer treatment group were 0.014 (day 8), 0.009 (day 12), 0.0009 (day 16), 0.000001 (day 27), 0.000004 (day 30), 0.00001 (day 33), and 0.00001 (day 36). In the right panel, the fold change in tumor volume for each tumor at day 36 is shown.

FIG. 14—“Survival” of Syrian hamsters with HaK tumors. Kaplan-Meier analysis of survival curves was performed in SPSS. Survival curves were generated using a tumor volume of 2.0 ml. The high dose treatment group (n=18) was significantly different from the other groups, as determined by a log-rank test (p<0.0001). The low dose treatment group (n=16) was not significantly different from the mock group (n=16) according to a log-rank test (p=0.0845). At day 36, 78% (14/18) of tumors in the high dose VRX-007 group had not reached the cutoff volume as compared to only 6% (1/16) of tumors in the mock group.

FIG. 15—Photographs of HaK subcutaneous tumors. Photographs were taken at necropsy of the HaK subcutaneous tumors of three animals each of the mock and high dose VRX-007 groups. The photographs show the hind end of animal. The hamsters on the top were injected with buffer, while those on the bottom received the high dose of VRX-007. The light gray areas show where the animals had been shaved. The left and right photographs are the same except that in the right photograph the tumors that were injected are outlined in black.

FIG. 16—Lung staining to visualize metastases. Upon necropsy, the lungs of hamsters in the HaK study were stained with India ink to more easily visualize metastases (upper panel). The lungs were scored on a scale of zero to five, with five being maximum tumor burden and zero being no grossly visible metastases. The mean rating for each group was plotted (lower panel). As shown in the photograph as well as the bar graph, the high dose of VRX-007 suppressed the degree of pulmonary metastasis by about 75 percent.

FIG. 17—Mean hamster weight gain/loss. Hamsters were all weighed on day 0 and surviving hamsters were weight on the days indicated in the text. The percent weight gain or loss with respect to day 0 was calculated for each hamster. The mean weight gain/loss was potted vs. day post injection.

FIGS. 18A-D—VRX-007 replication. VRX-007 replicates in LCRT tumors (FIG. 18A) but not in blood (FIG. 18B), liver (FIG. 18C), or lung (FIG. 18D) after intratumoral injection of a single dose of 1×10⁹ PFU of virus. The graphs depict the mean titers obtained from three samples, and the whiskers show standard deviation. Pre-established subcutaneous LCRT tumors (300-400 μl) in cotton rats (one tumor per animal) were injected with buffer or 1×10⁹ PFU of VRX-007. Groups of three animals were sacrificed by CO₂ asphyxiation at 4 h post injection, and then 2, 4, 6, 8, 10, and 13 days later. Before sacrificing the animals, blood was collected from the retro-orbital sinus. The rats that were sacrificed at 4 h post injection were bled at 30 min and 2 h post injection as well. All animals were exsanguinated to reduce contamination of organs with blood. Lungs, livers, and tumors were collected using sterile instruments, and snap frozen in liquid nitrogen. The tissues (full organs) were homogenized by fracturing the frozen organs in a Bio-Pulverizer device (Research Products International, Mount Prospect, IL). The samples were weighed, reconstituted with DMEM, and the cells were lysed by three cycles of freeze-thawing and 15 min of sonication. The samples were cleared by low-speed centrifugation and titered in an end-point dilution assay on A549 cells in 96 well plates, 12 parallels for each sample. The plates were read 14 days p.i., and the 50% Tissue Culture Infective Dose (TCID₅₀) was calculated.

FIG. 19—VRX-007 replicates in HaK tumors. A single dose of 6×10⁹ PFU (2.5×10¹¹ virus particles) of VRX-007 or 4×10⁸ PFU (2.5×10¹¹ virus particles) of Ad-EGFP was injected into pre-established subcutaneous HaK tumors. Tumors were harvested at 1.5 hours and 1, 2, 4, 7, and 14 days post injection and virus yields were determined by TCID₅₀ assay on 293 cells. The mean total virus yield per tumor is shown. Both VRX-007 and Ad-EGFP are able to replicate within and lyse 293 cells. 293 cells are an established human cell line that stably expresses the adenovirus E1A and E1B proteins. Expression of these proteins allow Ad-AGFP to grow in 293 cells.

FIGS. 20A-C—VRX-007, Ad5, and VRX-EGFP suppress the growth of subcutaneous HaK tumors in hamsters. Cells were injected inot the hind flanks of hamsters. When the tumors reached about 250 μl, they were injected with the indicated viruses for five consecutive days. Tumors were measured with digital calipers. The left figure (FIG. 20A) shows the mean volumes of 12 hamsters in each group. The, middle and right figures (FIGS. 20B and 20C) show individual tumors in the mock- and VRX-007-infected groups, respectively; the numbers refer to animal number. The tumor growth line ends when the hamster was euthanized. Some of the mock tumors decreased in size the last time point because they became ulcerated through the skin and released exudates.

### DESCRIPTION OF ILLUSTRATIVE EMBODIMENTS

A. The Present Invention

As discussed above, there remains a need to develop new and improved animal models for the testing of adenoviral vectors, particularly those that are replication competent. The present inventors have developed two such models, as discussed in detail below.

1. Cotton Rat

In addressing the aforementioned need for improved animal models, the inventors have developed an immunocompetent animal model to examine the efficacy of adenoviral vectors using syngeneic tumors in immunocompetent cotton rats. The cotton rat, a species more closely related to hamsters than rats, is an animal in which Ad5 has been shown to replicate in the lung and to cause pathology that resembles that seen in Ad5-infected humans (Pacini et al., 1984; Prince et al., 1993). Since this animal is immunocompetent and, at least partially a permissive host for adenovirus replication, the interaction of the host, the tumor, and the vector may be examined in its full complexity.

The cotton rat is an established model for studying the pathology of adenovirus, respiratory syncytial virus, poliovirus, typhus, and filariasis (www.nal.usda.gov/awic/newsletters/v5n2/5n2princ.htm). Furthermore, a study was done on cotton rats to test the toxicity, spread, and shedding of an oncolytic Ad vector upon subcutaneous injection (Wildner and Morris, 2002). However, inasmuch as no tumor was used, this latter study was of limited value.

The study of cotton rats as models for human diseases was pioneered by a company named Virion Systems, Inc. (Rockville, Md.), which later provided Harlan Sprague Dawley and Charles River with breeding colonies. Today, both laboratory animal suppliers sell inbred cotton rats that are descendants of the original colony obtained from Virion Systems. During their work with cotton rats, Virion Systems established a cell line named LCRT, from a spontaneous sarcoma tumor. This cell line can be cultured using routine tissue culture techniques and forms tumors when injected subcutaneously into cotton rats.

2. Syrian Hamster

In addition to cotton rats, human Ad has been reported to replicate in Syrian hamsters following intranasal inoculation (Hjorth et al., 1988; Khoobyarian et al., 1975; Morin et al., 1987). Therefore, investigation into the use of Syrian hamsters as a model for testing the efficacy of oncolytic Ad vectors was intiated. Two Syrian hamster cell lines were obtained from ATCC (HaK-a kidney cell line and DDT1 MF-2-a ductus deferens leiomyosarcoma cell line) as well as two Syrian hamster cell lines from a laboratory at the University of Nebraska Medical Center (PC1 and PC1.0-pancreatic carcinoma cell lines) (Egami et al., 1991). These cell lines were chosen based on their permissivity to other virus infections, their ability to form subcutaneous tumors in hamsters, and the presence of literature available on their use in hamsters.

3. Parameters for Examination

Assessing the efficacy of a therapeutic agent in pre-clinical models is crucial to successfully advancing the agent into clinical trials. The efficacy of adenoviruses is typically evaluated by determining their ability to affect the growth of human tumors. In models where the tumor is growing subcutaneously, efficacy may be assessed by measuring the size of the tumor, which can be performed using manual or digital calipers, or by non-invasive means such as scanning the tumor using a CT or ultrasound device. Alternatively, the agent's efficacy may be determined upon post-mortem gross pathological examination of the consistency, weight, and appearance of the tumor. Tumors excised at the time of necropsy may also be assessed by evaluating, the ability of the agent to induce tumor cell necrosis and apoptosis. These evaluations may be performed using techniques such as histopathological examination of stained sections, immunohistochemical procedures for detecting proteins expressed by the agent or associated with the processes of necrosis and apoptosis, in situ PCR or RT-PCR for detecting the agent or a gene induced by the agent, and in situ detection of apoptosis-related changes to cell. In models where the extent of metastasis is the endpoint, the number and size of tumor metastases can determined. In models where death of the animal is the endpoint, the agent's efficacy can be assessed using increase in survival as measured by percent of surviving animals on a particular day or the mean survival time of the animals.

The efficacy of the agent may also be evaluated by determining its ability to alter immune system parameters. Tumors and surrounding tissues may be evaluated for the extent of infiltration of immune mediators such as T-cells, macrophages, dendritic cells, B-cells, NK cells, NKT cells, nuetrophils, and other cells of the immune system. These cells may be identified using histochemical, immunohistochemical, or functional assays. The induction of cytokines and chemokines can also be measured using assays that measure the functional or physical amount of the molecule. Measurement of antibodies directed against the agent or the tumor can also be used to determine the efficacy of the agent.

Where the adenovirus expresses a therapeutic protein, efficacy may be assessed by measuring the effect of the therapeutic protein on the tumor and surrounding tissue. For example, if the agent is an angiogenic antagonist, efficacy may be appraised by measuring the inhibition of de novo vascularization.

The efficacy of an agent is determined not only by its ability to destroy tumor cells, but also by its ability to leave normal tissues intact. Thus, the efficacy of the agent may also be measured by determining the extent to which normal tissues are affect by the agent. This may be assessed by histochemical or immunohistochemical staining of tissue sections, by assessing necrosis and apoptosis in normal tissues using the means described above, by evaluating blood analytes through clinical chemistry test, and by hematological alterations as determined by blood counts.

Furthermore, oncolytic Ads are replication-competent, and thus the efficacy of these agents may be assessed by determining the extent to which they replicate. Assays to evaluate this parameter include determine the amount of infectious virus or vector in the blood and/or tissue, measuring the amount viral mRNA and/or genomic DNA in tissue and blood samples, assessing tissue samples for expression of viral proteins using various immunological techniques such as immunohistochemistry, western blotting, FACS analysis, and ELISA.

B. Adenovirus

1. Virus Characteristics

Adenovirus is a non-enveloped double-stranded DNA virus. The virion consists of a DNA-protein core within a protein capsid. Virions bind to a specific cellular receptor, are endocytosed, and the genome is extruded from endosomes and transported to the nucleus. The genome is about 36 kB, encoding about 36 genes. In the nucleus, the “immediate early” E1A proteins are expressed initially, and these proteins induce expression of the “delayed early” proteins encoded by the E1B, E2, E3, and E4 transcription units. Virions assemble in the nucleus at about 1 day post infection (p.i.), and after 2-3 days the cell lyses and releases progeny virus. Cell lysis is mediated by the E3 11.6K protein, which has been renamed “adenovirus death protein” (ADP).

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

Adenovirus may be any of the 51 different known serotypes or subgroups A-F. Adenovirus type 5 of subgroup C is the human adenovirus about which the most biochemical and genetic information is known, and it has historically been used for most constructions employing adenovirus as a vector. Recombinant adenovirus often is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Viruses used in gene therapy may be either replication-competent or replication-deficient. Generation and propagation of the adenovirus vectors which are replication-deficient depends on a helper cell line, the prototype being 293 cells, prepared by transforming human embryonic kidney cells with Ad5 DNA fragments; this cell line constitutively expresses E1 proteins (Graham et al., 1977). However, helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Adenovirus growth and manipulation is known to those of skill in the art, and exhibits broad host range in vitro and in vivo. This group of viruses can be obtained in high titers, e.g., 10⁹-10¹³ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

2. Engineering

As stated above, Ad vectors are based on recombinant Ad's that are either replication-defective or replication-competent. Typical replication-defective Ad vectors lack the E1A and E1B genes (collectively known as E1) and contain in their place an expression cassette consisting of a promoter and pre-mRNA processing signals which drive expression of a foreign gene. These vectors are unable to replicate because they lack the E1A genes required to induce Ad gene expression and DNA replication. In addition, the E3 genes can be deleted because they are not essential for virus replication in cultured cells. It is recognized in the art that replication-defective Ad vectors have several characteristics that make them suboptimal for use in therapy. For example, production of replication-defective vectors requires that they be grown on a complementing cell line that provides the E1A proteins in trans.

Several groups have also proposed using replication-competent Ad vectors for therapeutic use. Replication-competent vectors retain Ad genes essential for replication, and thus do not require complementing cell lines to replicate. Replication-competent Ad vectors lyse cells as a natural part of the life cycle of the vector. An advantage of replication-competent Ad vectors occurs when the vector is engineered to encode and express a foreign protein. Such vectors would be expected to greatly amplify synthesis of the encoded protein in vivo as the vector replicates. For use as anti-cancer agents, replication-competent viral vectors would theoretically be advantageous in that they would replicate and spread throughout the tumor, not just in the initially infected cells as is the case with replication-defective vectors.

Yet another approach is to create viruses that are conditionally-replication competent. Onyx Pharmaceuticals recently reported on adenovirus-based anti-cancer vectors which are replication-deficient in non-neoplastic cells, but which exhibit a replication phenotype in neoplastic cells lacking functional p53 and/or retinoblastoma (pRB) tumor suppressor proteins (U.S. Pat. No. 5,677,178). This phenotype is reportedly accomplished by using recombinant adenoviruses containing a mutation in the E1B region that renders the encoded E1B-55K protein incapable of binding to p53 and/or a mutation(s) in the E1A region which make the encoded E1A protein (p289R or p243R) incapable of binding to pRB and/or p300 and/or p107. E1B-55K has at least two independent functions: it binds and inactivates the tumor suppressor protein p53, and it is required for efficient transport of Ad mRNA from the nucleus. Because these E1B and E1A viral proteins are involved in forcing cells into S-phase, which is required for replication of adenovirus DNA, and because the p53 and pRB proteins block cell cycle progression, the recombinant adenovirus vectors described by Onyx should replicate in cells defective in p53 and/or pRB, which is the case for many cancer cells, but not in cells with wild-type p53 and/or pRB.

Another replication-competent adenovirus vector has the gene for E1B-55K replaced with the herpes simplex virus thymidine kinase gene (Wilder et al., 1999a). The group that constructed this vector reported that the combination of the vector plus gancyclovir showed a therapeutic effect on a human colon cancer in a nude mouse model (Wilder et al., 1999b). However, this vector lacks the gene for ADP, and accordingly, the vector will lyse cells and spread from cell-to-cell less efficiently than an equivalent vector that expresses ADP.

The present inventor has taken advantage of the differential expression of telomerase in dividing cells to create novel adenovirus vectors which overexpress an adenovirus death protein and which are replication-competent in and, preferably, replication-restricted to cells expressing telomerase. Specific embodiments include disrupting E1A's ability to bind p30⁰ and/or members of the Rb family members. Others include Ad vectors lacking expression of at least one E3 protein selected from the group consisting of 6.7K, gp19K, RIDβ (also known as 10.4K); RIDE (also known as 14.5K) and 14.7K. Because wild-type E3 proteins inhibit immune-mediated inflammation and/or apoptosis of Ad-infected cells, a recombinant adenovirus lacking one or more of these E3 proteins may stimulate infiltration of inflammatory and immune cells into a tumor treated with the adenovirus and that this host immune response will aid in destruction of the tumor as well as tumors that have metastasized. A mutation in the E3 region would impair its wild-type function, making the viral-infected cell susceptible to attack by the host's immune system. These viruses are described in detail in U.S. Pat. No. 6,627,190.

Other adenoviral vectors are described in U.S. Pat. Nos. 5,670,488; 5,747,869; 5,981,225; 6,069,134; 6,136,594; 6,143,290; 6,410,010; and 6,511,184.

C. Therapeutic Genes

In accordance with certain aspects of the present invention, Ad vectors carrying therapeutic genes will be screened for their ability to confer therapeutic benefit of the animal models disclosed herein. Virtually any therapeutic nucleic acid may be used, and the following provide non-limiting examples of these.

1. Antisense Nucleic Acids

As used herein, the terms “antisense” or “complementary” mean nucleic acids that are substantially complementary over their entire length and have very few base mismatches. The nucleic acids may be DNA or RNA molecules. A nucleic acid “complement(s)” or is “complementary” to another nucleic acid when it is capable of base-pairing with another nucleic acid according to the standard Watson-Crick, Hoogsteen or reverse Hoogsteen binding complementarity rules.

The polynucleotides according to the present invention may correspond to a particular gene or portion of that gene sufficient to effect antisense inhibition of transcription or translation. The polynucleotides may be derived from genomic DNA, i.e., cloned directly from the genome of a particular organism. In other embodiments, however, the polynucleotides may be complementary DNA (cDNA). cDNA is DNA prepared using messenger RNA (mRNA) as template. Thus, a cDNA does not contain any interrupted coding sequences and usually contains almost exclusively the coding region(s) for the corresponding protein. In other embodiments, the antisense polynucleotide may be produced synthetically.

It may be advantageous to combine portions of the genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

Antisense sequences may be full length genomic or cDNA copies, or large fragments thereof, or may be shorter fragments, or “oligonucleotides,” defined herein as polynucleotides of 50 or less bases. Although shorter oligomers (8-20) are easier to make and increase in vivo accessibility, numerous other factors are involved in determining the specificity of base-pairing. For example, both binding affinity and sequence specificity of an oligonucleotide to its complementary target increase with increasing length. It is contemplated that oligonucleotides of 8, 9, 10, 11, 12, 13, 14, 15, 16, 17, 18, 19, 20, 25, 30, 35, 40, 45 or 50 base pairs will be used. While all or part of the gene sequence may be employed in the context of antisense construction, statistically, any sequence of 17 bases long should occur only once in the human genome and, therefore, suffice to specify a unique target sequence.

In certain embodiments, one may wish to employ antisense constructs which include other elements, for example, those which include C-5 propyne pyrimidines. Oligonucleotides which contain C-5 propyne analogues of uridine and cytidine have been shown to bind RNA with high affinity and to be potent antisense inhibitors of gene expression (Wagner et al., 1993).

Alternatively, the antisense oligo- and polynucleotides according to the present invention may be provided as mRNA via transcription from expression constructs that carry nucleic acids encoding the oligo- or polynucleotides. Throughout this application, the term “expression construct” is meant to include any type of genetic construct containing a nucleic acid encoding an antisense product in which part or all of the nucleic acid sequence is capable of being transcribed. Typical expression vectors include bacterial plasmids or phage, such as any of the pUC or Bluescrip™ plasmid series or, as discussed further below, viral vectors adapted for use in eukaryotic cells.

Typical targets include genes that can trigger cancer development. These genes are often called “oncogenes.” Table 0 lists some of these targets.

TABLE 0
Oncogenes

| | | | |
|---|---|---|---|
| Gene | Source | Human Disease | Function |

Growth Factors

| | | |
|---|---|---|
| HST/KS | Transfection | FGF family member |
| INT-2 | MMTV promoter | FGF family member |
| | Insertion | |
| INTI/WNTI | MMTV promoter | Factor-like |
| | Insertion | |
| SIS | Simian sarcoma virus | PDGF B |

Receptor Tyrosine Kinases

| | | | |
|---|---|---|---|
| ERBB/HER | Avian erythroblastosis | Amplified, deleted | EGF/TGF-α/ |
| | virus; ALV promoter | squamous cell | amphiregulin/ |
| | insertion; amplified | cancer; glioblastoma | hetacellulin receptor |
| | human tumors | | |
| ERBB-2/NEU/HER-2 | Transfected from rat | Amplified breast, | Regulated by NDF/ |
| | Glioblatoms | ovarian, gastric cancers | heregulin and EGF- |
| | | | related factors |
| FMS | SM feline sarcoma virus | | CSF-1 receptor |
| KIT | HZ feline sarcoma virus | | MGF/Steel receptor |
| | | | hematopoieis |
| TRK | Transfection from | | NGF (nerve growth |
| | human colon cancer | | factor) receptor |
| MET | Transfection from | | Scatter factor/HGF |
| | human osteosarcoma | | receptor |
| RET | Translocations and point | Sporadic thyroid cancer; | Orphan receptor Tyr |
| | mutations | familial medullary | kinase |
| | | thyroid cancer; | |
| | | multiple endocrine | |
| | | neoplasias 2A and 2B | |
| ROS | URII avian sarcoma | | Orphan receptor Tyr |
| | Virus | | kinase |
| PDGF receptor | Translocation | Chronic | TEL(ETS-like |
| | | myclomonocytic | transcription factor)/ |
| | | leukemia | PDGF receptor gene |
| | | | fusion |
| TGF-β receptor | | Colon carcinoma | |
| | | mismatch mutation | |
| | | target | |

NONRECEPTOR TYROSINE KINASES

| | | | |
|---|---|---|---|
| ABI. | Abelson Mul. V | Chronic myelogenous | Interact with RB, RNA |
| | | leukemia translocation | polymerase, CRK, |
| | | with BCR | CBL |
| FPS/FES | Avian Fujinami SV; GA | | |
| | FeSV | | |
| LCK | Mul. V (murine leukemia | | Src family; T cell |
| | virus) promoter | | signaling; interacts |
| | insertion | | CD4/CD8 T cells |
| SRC | Avian Rous sarcoma | | Membrane-associated |
| | Virus | | Tyr kinase with |
| | | | signaling function; |
| | | | activated by receptor |
| | | | kinases |
| YES | Avian Y73 virus | | Src family; signaling |

SER/THR PROTEIN KINASES

| | | |
|---|---|---|
| AKT | AKT8 murine retrovirus | Regulated by PI(3)K?; |
| | | regulate 70-kd S6 k? |
| MOS | Maloney murine SV | GVBD; cystostatic |
| | | factor; MAP kinase |
| | | kinase |
| PIM-1 | Promoter insertion | |
| | Mouse | |
| RAF/MIL | 3611 murine SV; MH2 | Signaling in RAS |
| | avian SV | pathway |

MISCELLANEOUS CELL SURFACE

| | | | |
|---|---|---|---|
| APC | Tumor suppressor | Colon cancer | Interacts with catenins |
| DCC | Tumor suppressor | Colon cancer | CAM domains |
| E-cadherin | Candidate tumor | Breast cancer | Extracellular homotypic |
| | Suppressor | | binding; intracellular |
| | | | interacts with catenins |
| PTC/NBCCS | Tumor suppressor and | Nevoid basal cell cancer | 12 transmembrane |
| | Drosophilia homology | syndrome (Gorline | domain; signals |
| | | syndrome) | through Gli homogue |
| | | | CI to antagonize |
| | | | hedgehog pathway |
| TAN-1 Notch | Translocation | T-ALI. | Signaling? |
| homologue | | | |

MISCELLANEOUS SIGNALING

| | | | |
|---|---|---|---|
| BCL-2 | Translocation | B-cell lymphoma | Apoptosis |
| CBL | Mu Cas NS-1 V | | Tyrosine- |
| | | | phosphorylated RING |
| | | | finger interact Abl |
| CRK | CT1010 ASV | | Adapted SH2/SH3 |
| | | | interact Abl |
| DPC4 | Tumor suppressor | Pancreatic cancer | TGF-β-related signaling |
| | | | pathway |
| MAS | Transfection and | | Possible angiotensin |
| | Tumorigenicity | | receptor |
| NCK | | | Adaptor SH2/SH3 |

GUANINE NUCLEOTIDE EXCHANGERS AND BINDING PROTEINS

| | | | |
|---|---|---|---|
| BCR | | Translocated with ABL | Exchanger; protein |
| | | in CML | kinase |
| DBL | Transfection | | Exchanger |
| GSP | | | |
| NF-1 | Hereditary tumor | Tumor suppressor | RAS GAP |
| | Suppressor | neurofibromatosis | |
| OST | Transfection | | Exchanger |
| Harvey-Kirsten, N-RAS | HaRat SV; Ki RaSV; | Point mutations in many | Signal cascade |
| | Balb-MoMuSV; | human tumors | |
| | Transfection | | |
| VAV | Transfection | | S112/S113; exchanger |

NUCLEAR PROTEINS AND TRANSCRIPTION FACTORS

| | | | |
|---|---|---|---|
| BRCA1 | Heritable suppressor | Mammary | Localization unsettled |
| | | cancer/ovarian cancer | |
| BRCA2 | Heritable suppressor | Mammary cancer | Function unknown |
| ERBA | Avian erythroblastosis | | thyroid hormone |
| | Virus | | receptor (transcription) |
| ETS | Avian E26 virus | | DNA binding |
| EVII | MuLV promoter | AML | Transcription factor |
| | Insertion | | |
| FOS | FBI/FBR murine | | 1 transcription factor |
| | osteosarcoma viruses | | with c-JUN |
| GLI | Amplified glioma | Glioma | Zinc finger; cubitus |
| | | | interruptus homologue |
| | | | is in hedgehog |
| | | | signaling pathway; |
| | | | inhibitory link PTC |
| | | | and hedgehog |
| HMGG/LIM | Translocation t(3:12) | Lipoma | Gene fusions high |
| | t(12:15) | | mobility group |
| | | | HMGI-C (XT-hook) |
| | | | and transcription factor |
| | | | LIM or acidic domain |
| JUN | ASV-17 | | Transcription factor |
| | | | AP-1 with FOS |
| MLL/VHRX + ELI/MEN | Translocation/fusion | Acute myeloid leukemia | Gene fusion of DNA- |
| | ELL with MLL | | binding and methyl |
| | Trithorax-like gene | | transferase MLL with |
| | | | ELI RNA pol II |
| | | | elongation factor |
| MYB | Avian myeloblastosis | | DNA binding |
| | Virus | | |
| MYC | Avian MC29; | Burkitt's lymphoma | DNA binding with |
| | Translocation B-cell | | MAX partner; cyclin |
| | Lymphomas; promoter | | regulation; interact |
| | Insertion avian | | RB?; regulate |
| | leukosis | | apoptosis? |
| | Virus | | |
| N-MYC | Amplified | Neuroblastoma | |
| L-MYC | | Lung cancer | |
| REL | Avian | | NF-κB family |
| | Retriculoendotheliosis | | transcription factor |
| | Virus | | |
| SKI | Avian SKV770 | | Transcription factor |
| | Retrovirus | | |
| VHL | Heritable suppressor | Von Hippel-Landau | Negative regulator or |
| | | syndrome | elongin; transcriptional |
| | | | elongation complex |
| WT-1 | | Wilm's tumor | Transcription factor |

CELL CYCLE/DNA DAMAGE RESPONSE

| | | | |
|---|---|---|---|
| ATM | Hereditary disorder | Ataxia-telangiectasia | Protein/lipid kinase |
| | | | homology; DNA |
| | | | damage response |
| | | | upstream in P53 |
| | | | pathway |
| BCL-2 | Translocation | Follicular lymphoma | Apoptosis |
| FACC | Point mutation | Fanconi's anemia group | |
| | | C (predisposition | |
| | | leukemia | |
| FHIT | Fragile site 3p14.2 | Lung carcinoma | Histidine triad-related |
| | | | diadenosine 5′,3″″- |
| | | | P¹.p⁴ tetraphosphate |
| | | | asymmetric hydrolase |
| hMLI/MutL | | HNPCC | Mismatch repair; MutL |
| | | | homologue |
| hMSH2/MutS | | HNPCC | Mismatch repair; MutS |
| | | | homologue |
| hPMS1 | | HNPCC | Mismatch repair; MutL |
| | | | homologue |
| hPMS2 | | HNPCC | Mismatch repair; MutL |
| | | | homologue |
| INK4/MTS1 | Adjacent INK-4B at | Candidate MTS1 | p16 CDK inhibitor |
| | 9p21; CDK complexes | suppressor and MLM | |
| | | melanoma gene | |
| INK4B/MTS2 | | Candidate suppressor | p15 CDK inhibitor |
| MDM-2 | Amplified | Sarcoma | Negative regulator p53 |
| p53 | Association with SV40 | Mutated >50% human | Transcription factor; |
| | T antigen | tumors, including | checkpoint control; |
| | | hereditary Li-Fraumeni | apoptosis |
| | | syndrome | |
| PRAD1/BCL1 | Translocation with | Parathyroid adenoma; | Cyclin D |
| | Parathyroid hormone | B-CLL | |
| | or IgG | | |
| RB | Hereditary | Retinoblastoma; | Interact cyclin/cdk; |
| | Retinoblastoma; | osteosarcoma; breast | regulate E2F |
| | Association with many | cancer; other sporadic | transcription factor |
| | DNA virus tumor | cancers | |
| | Antigens | | |
| XPA | | xeroderma | Excision repair; photo- |
| | | pigmentosum; skin | product recognition; |
| | | cancer predisposition | zinc finger |

2. Ribozymes

Another general class of inhibitors is ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphodiester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). It has also been shown that ribozymes can elicit genetic changes in some cells lines to which they were applied; the altered genes included the oncogenes H-ras, c-fos and genes of HIV. Most of this work involved the modification of a target mRNA, based on a specific mutant codon that was cleaved by a specific ribozyme. These molecules may be used to target any of the genes listed in Table 0, or other suitable oncogenes.

3. siRNA

RNA interference (also referred to as “RNA-mediated interference” or RNAi) is another mechanism by which oncogene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fuingi, C. elegans, Trypanasoma, Drosophila, and mammals (Grishok et al., 2000; Sharp, 1999; Sharp et al., 2000; Elbashir et al., 2001). It is generally accepted that RNAi acts post-transcriptionally, targeting RNA transcripts for degradation. It appears that both nuclear and cytoplasmic RNA can be targeted (Bosher et al., 2000).

siRNAs must be designed so that they are specific and effective in suppressing the expression of the genes of interest. Methods of selecting the target sequences, i.e. those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,732, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM. This had been demonstrated by Elbashir et al. (2001) wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single stranded RNA is enzymatically synthesized from the PCR™ products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

4. Proteins

The term “gene” is used for simplicity to refer to a functional protein, polypeptide, or peptide-encoding unit. “Therapeutic gene” is a gene that can be administered to a subject for the purpose of treating or preventing a disease. For example, a therapeutic gene can be a gene administered to a subject for treatment or prevention of cancer. Examples of classes of therapeutic genes include tumor suppressors, inducers of apoptosis, cell cycle regulators, toxins, cytokines, enzymes, antibodies, inhibitors of angiogenesis, metalloproteinase inhibitors, hormones or peptide hormones. Examples of therapeutic genes include, but are not limited to, Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIMI, PML, RET, SRC, TAL1, TCL3, YES, MADH4, RB1, TP53, WT1, TNF, BDNF, CNTF, NGF, IGF, GMF, aFGF, bFGF, NT3, NT5, ApoAl, ApoAIV, ApoE, RaplA, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, zac1, DBCCR-1, rks-3, COX-1, TFPI, PGS, Dp, E2F, E1A, p300/CBP, VEGF, FGF, thrombospondin, BAI-1, GDAIF, MCC, Bax, Bad, Bid, Bik, Bcl-Xₛ, HSV-tk, TRAIL, IL-1 to 18 and 24.

D. Combination Therapies

In order to increase the effectiveness of a Ad-based cancer therapy, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other therapies would be provided in a combined amount effective to kill or inhibit proliferation of the cell. This process may involve contacting the cells with the Ad vector and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the Ad vector and the other includes the second agent(s).

Alternatively, the Ad vector may precede or follow the other therapy by intervals ranging from minutes to weeks. In embodiments where the agents are administered separately to the subject, one would generally ensure that a significant period of time did not expire between the time of each delivery, such that the two therapies would still be able to exert an advantageous effect on the subject. In such instances, it is contemplated that one may administer both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, the Ad vector is “A” and the other agent is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is expected that the treatment cycles would be repeated as necessary. It also is contemplated that various secondary forms of therapy, such as surgical intervention, chemotherapy, or radiotherapy, hormonal therapy, immunotherapy, or a second gene therapy, may be applied in combination with the described Ad vector. For example, these secondary therapies may be applied in combination with the Ad vector to treat a patient with cancer.

1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristin, vinblastin and methotrexate, or any analog or derivative variant of the foregoing.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

3. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with Ad gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

4. Genes

In yet another embodiment, the secondary treatment is a secondary gene therapy in which a second therapeutic polynucleotide is administered before, after, or at the same time a first therapeutic polynucleotide. Delivery of an Ad vector encoding either a first and second gene, or two vectors, each encoding a different gene, may be used. The genes may be selected from those listed above or others.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscropically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

E. Pharmaceutical Preparations and Therapeutic Methods

In certain embodiments of the present invention, the Ad vectors and other agents are presented in a pharmaceutically acceptable carrier or aqueous medium. The phrases “pharmaceutical preparation” or “pharmacologically effective” or “pharmaceutically acceptable” refer to molecular entities and compositions that do not produce an adverse, allergic or other untoward reaction when administered to an animal, or a human, as appropriate.

Certain embodiments of the present compositions include any and all solvents, dispersion media, coatings, antibacterial and antifingal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The biological material should be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, sub-cutaneous, intralesional, or even intraperitoneal routes. The preparation of an aqueous composition containing an active agent of the invention disclosed herein as a component or active ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An agent or substance of the present invention can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. In terms of using peptide therapeutics as active ingredients, the technology of U.S. Pat. Nos. 4,608,251; 4,601,903; 4,599,231; 4,599,230; 4,596,792; and 4,578,770, each incorporated herein by reference, may be used.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15^{th} Edition, pages 1035-1038 and 1570-1580). Some variation in dosage will necessarily occur depending on the condition of the subject being treated. The person responsible for administration will, in any event, determine the appropriate dose for the individual subject.

The active agents disclosed herein may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used, including cremes.

One may also use nasal solutions or sprays, aerosols or inhalants in the present invention. Nasal solutions are usually aqueous solutions designed to be administered to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

Additional formulations which are suitable for other modes of administration include vaginal suppositories and pessaries.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. An effective amount of the therapeutic or preventive agent is determined based on the intended goal, for example, prevention or reduction of inflammation secondary to administration of a lipid-nucleic acid complex to a subject. The quantity to be administered, both according to number of treatments and dose, depends on the subject to be treated, the state of the subject and the protection desired. Precise amounts of the therapeutic composition also depend on the judgment of the practitioner and are peculiar to each individual.

In certain embodiments, it may be desirable to provide a continuous supply of the therapeutic compositions to the patient. For topical administrations, repeated application would be employed. For various approaches, delayed release formulations could be used that provide limited but constant amounts of the therapeutic agent over an extended period of time. For internal application, continuous perfusion of the region of interest may be preferred. This could be accomplished by catheterization, post-operatively in some cases, followed by continuous administration of the therapeutic agent. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the doses are administered.

### F. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Cotton Rat

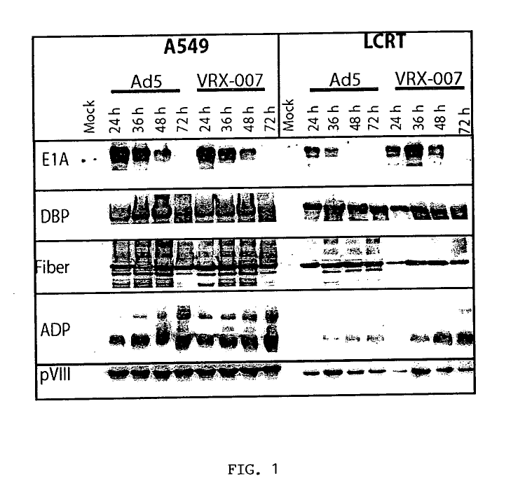

VRX-007 productively infects cultured LCRT cotton rat cells. VRX-007 is a recombinantly-engineered replication-competent Ad5-derived vector that overxpresses ADP in relation to wild-type Ad5. The inventors have demonstrated that Ad5 and VRX-007 infect, replicate in, and destroy LCRT cells in tissue culture. Three sets of experiments were performed to obtain this evidence. The first set of experiments assessed the ability of Ad5 and VRX-007 to establish an infection and then enter into the late phase of infection in the LCRT cell line. Lysates from infected cells were prepared and analyzed by western blot for the expression of a variety of Ad-encoded proteins that are expressed during the early or late phases of infection. For comparison, the inventors also prepared lysates from infected human A549 cells, a cell line that is permissive for Ad infection. The results show that, in LCRT cotton rat cells, the early proteins E1A and DBP are expressed at a level and in a time frame similar to that seen in A549 cells (FIG. 1). Late protein expression (i.e., Fiber, ADP, and protein pVIII) in LCRT cells was only slightly reduced compared to A549 cells (FIG. 1). These results hold true for infection with either Ad5 or VRX-007. These data demonstrate that Ads can establish an infection in the LCRT-cell line and that the infection efficiently enters the late phase.

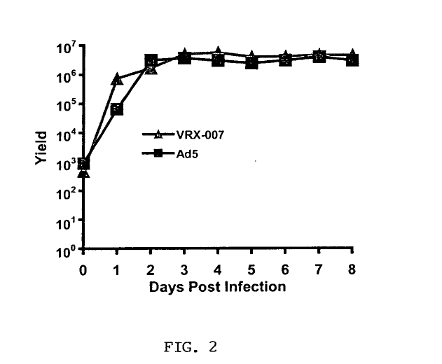

The data described above do not address if infection of this cotton rat cell line is productive (i.e., progeny virus is produced). In the second set of experiments, the inventors investigated the cell line's ability to produce progeny virus upon infection by performing a single step growth analysis. LCRT cells were infected with Ad5 or VRX-007, and the cells and the medium were harvested at given intervals after infection. Virus yield in these samples was determined by titering on human A549 cells. The titer of the LCRT samples increased with time (FIG. 2). This signifies that LCRT cells are permissive for adenovirus replication.

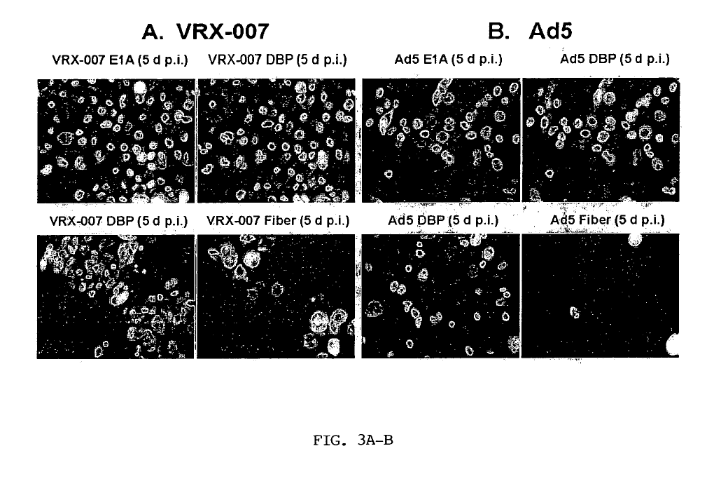

The third set of experiments was aimed at establishing whether adenoviruses could spread on the LCRT cotton rat cell line (i.e., to determine whether progeny virus released from infected cells can subsequently infect uninfected cells). LCRT cells grown on cover slips were infected at low multiplicities (so that only a few solitary cells get infected) of Ad5 or VRX-007. Five days after infection an indirect immunofluorescence assay was performed in order to detect the presence of adenovirus proteins in the cells. The presence of clusters of infected cells indicated that the viruses that were released from the originally infected cell established new infections in nearby cells (FIG. 3). These three experiments establish that VRX-007 and Ad5 grow well in LCRT cotton rat cells. They also suggest that the LCRT cell line may be a suitable cell line to test the efficacy of our vectors in cotton rats.

VRX-007 suppresses the growth of LCRT tumors grown in cotton rats. In a pilot experiment, it was established that LCRT cells form subcutaneous tumors in cotton rats by about 7-10 days after subcutaneous injection of 8×10⁶ cells (data not shown). The animals tolerated the tumors well, and did not show visible discomfort even when the tumors reached quite a large size (up to 20 ml). Furthermore, in this pilot experiment, we detected a putative effect after the intratumoral injection of VRX-007 (the effect could not be statistically evaluated because of the small number of animals used). Through this pilot experiment, the inventors gained some valuable experience as to how to handle these animals. This is not a trivial issue, as cotton rats are not domesticated and have very strong flight-or-fight instincts.

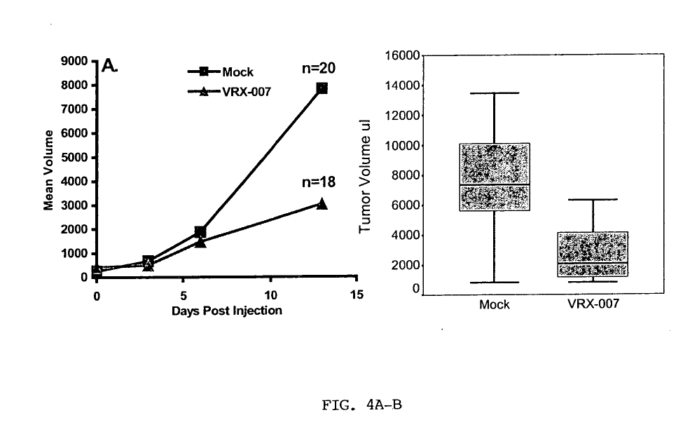

Following the pilot experiment, the inventors began additional experiments to determine the effect of VRX-007 treatment on the growth of LCRT tumors in cotton rats. In one experiment, subcutaneous LCRT tumors were established in cotton rats and then injected with ten doses of VRX-007 (8.5×10¹⁰ VP per dose) over a twelve-day span. The results show that over time LCRT tumors injected with VRX-007 were smaller than those tumors that were injected with buffer (FIG. 4A). VRX-007 treatment resulted in reduced median volume (FIG. 4B). In addition, the distribution of tumor sizes was skewed towards lower volumes indicating that in general the VRX-007-injected tumors did not grow as quickly (FIG. 4B). LCRT tumors are very aggressive when grown in cotton rats. During the course of the experiment, new, rapidly growing tumor nodules grew in areas surrounding the tumors that received the VRX-007 injections. This occurred even though the tumors that were injected with VRX-007 showed reduced growth. Gross pathological examination of the VRX-007-treated animals revealed that the “new” tumor nodules were encapsulated, which might explain why they grew despite the presence of VRX-007. Gross pathology also showed that both groups of animals contained metastases to the lung and mesenteric lymph nodes at an equal level. Despite the very aggressive nature of the LCRT tumors, the VRX-007 treatment resulted in two objective tumor responses while all of the buffer-injected tumors shrunk continued to grow rapidly.

A third experiment with subcutaneous LCRT tumors was performed. In this experiment, in addition to the buffer control, a control with UV inactivated VRX-007 also was performed. Cotton rats (4-5 weeks old females, 9 animals per group) were injected subcutaneously into both hind flanks with 1×10⁶ LCRT cells. The injection site was marked by a circle. Immediately after injection of cells, the injection site was injected with 50 μl of Lactated Ringer's Injection solution, USP (“mock”), 8.5×10¹⁰ VP of UV inactivated VRX-007 (“UV inactivated”), or intact VRX-007 (“VRX-007”). Injections were repeated daily for four consecutive days for a total dose of 3.4×10¹¹ VP. Virus was inactivated by irradiation with about 375 millijoules of ultraviolet light. The inactivated virus was tested in an end-point dilution assay, and was shown to contain about 1 viable particle per 6×10⁷ VP (data not shown).

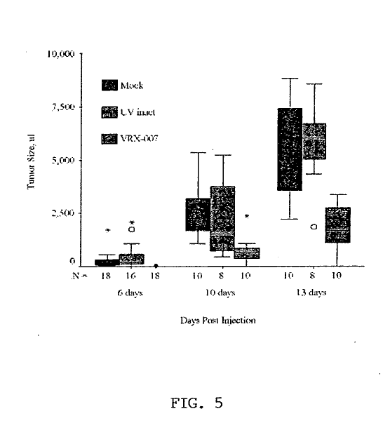

Six days after injection of cells the tumors were measured. Almost all tumors were visible by this time in the mock and UV-inactivated groups (18/18 and 16/18, mean volumes of 288 μl and 499 μl, respectively), but only a small fraction (3/18, mean volume: 10 μl) of injections resulted in detectable tumors in the VRX-007-treated group (FIG. 5). At this time, four animals from each group were sacrificed and necropsied. Gross pathological observations confirmed the data obtained by measuring the tumors, that is, no tumors were seen in the four VRX-007-injected animals, while all animals in the mock and UV inactivated groups had tumors on both sides. Samples of lungs, livers and injection sites were fixed in 10% formalin for histopathological and immunohistochemical analysis or were snap-frozen in liquid nitrogen for detection of viral nucleic acids by PCR and RT-PCR.

Tumors were measured again on days 10 and 13 post injection (FIG. 5). By day 13 post injection, tumors in the mock and UV inactivated groups grew to mean volumes of 5613 μl and 5742 μl, respectively, which exemplifies the fast growth characteristic of LCRT tumors. The mean volume of VRX-007-injected tumors was only 1772 μl, which represents a 68% inhibition of tumor growth (compared to the mock group). After the last measurement, the remaining animals were sacrificed and necropsied. All animals except one in the VRX-007 group had tumors at the injection site; this latter animal developed a tumor about 15 mm distance from the injection site. The tumors in the VRX-007 group were generally smaller than tumors in the other two groups, which is in agreement with the tumor volumes measured in live animals. Three out of five cotton rats in the mock group had metastases in their inguinal and lumbar lymph nodes. No metastases were seen in any animals in the other two groups. Histopathological examination of tissue sections showed that there were no differences in lung or liver sections between any of the treatment groups. Histopathology did show that tumors harvested on day 13 were more vascularized than those harvested on day 6. In addition, most tumors harvested day 6 or 13 showed signs of calcification and the presence of giant cells. Samples for immunohistochemical, PCR, and RT-PCR analysis were collected as for the earlier time point.

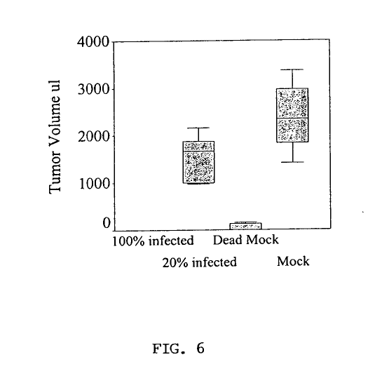

An experiment was also performed in which the LCRT cells used to establish the tumors in cotton rats were seeded with VRX-007-infected cells. LCRT cells in culture dishes were incubated with VRX-007 at 100 PFU/cell at 37° C. for 1 h, washed with PBS three times and harvested by trypsinization. These cells were termed “100% infected”. A portion of these cells was mixed with uninfected LCRT cells, harvested by trypsinization, at a ratio of one to five. This sample was termed “20% infected”. In addition, uninfected LCRT cells were trypsinized and split into two batches. One batch was labeled “mock”, the other was frozen and thawed three times and labeled “dead mock.” Groups of three cotton rats were injected subcutaneously into both hind flanks with one of the samples. With respect to tumor sizes, a clear difference between mock and “100% infected groups” was evident at twelve days post injection (FIG. 6). Even when only 20% of cells were pre-infected with VRX-007, tumor growth was still significantly reduced (p<0.05, Student's t test).

Cumulatively, these experiments with LCRT cells and cotton rats demonstrate that VRX-007 productively infects LCRT cells and that VRX-007 is effective at suppressing the growth of LCRT tumors grown in cotton rats, even though these tumors are very aggressive.

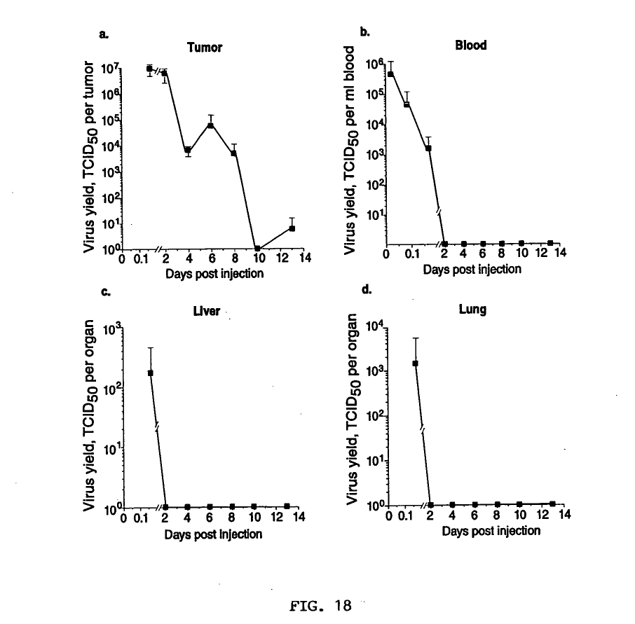

VRX-007 replicates in subcutaneous LCRT tumors. The anti-tumor effect of oncolytic Ad vectors is attributed to virus replication in and lysis of infected tumor cells. To address whether VRX-007 replicates in the LCRT tumors or in non-tumor tissues of cotton rats, we sampled blood, lung, liver, and tumor tissue at various times after a single intratumoral injection of VRX-007. VRX-007 particles were extracted and viable virus was titered. In the tumor samples, high titers of viable virus were obtained 4 h and 2 days after injection, followed by a drop in titer of three orders of magnitude by day 4 (FIG. 18A). A secondary peak of virus was detected on day 6 and then titers declined again to undetectable levels by day 10. Conversely, Ad in the blood decreased rapidly, and was not detected after two days (FIG. 18B). Results similar to those in blood were obtained with liver and lung samples (FIGS. 18C, 18D).

Altogether, these results indicate that VRX-007 replicated in tumors but not in the normal tissues. It is not likely that VRX-007 merely persisted in the tumors without replicating, because an earlier study showed that replication-defective Ads do not persist in cotton rats as infectious particles (Rojas-Martinez et al., 1998). Lack of VRX-007 replication in the lung and liver was not surprising considering that intramuscular (Oualikene et al., 1994) or subcutaneous (Wildner et al., 2002) injection of a replication-competent Ad into cotton rats did not lead to infection of the lung or liver. Also, cotton rats tolerated the intravenous injection of 1.4×10¹² virus particle (vp)/kg of VRX-007 with only transient morbidity (data not shown).

Acute toxicity in immunocompetent cotton rats following single-dose intravenous administration of VRX-007. Because Ad5 should replicate in cotton rats, the inventors next investigated the maximum tolerated dose of VRX-007 in this animal model. Three animals per group were injected one time into the jugular vein with 100 μl containing 1.7×10¹¹, 1.7×10¹⁰, or 1.7×10⁹ particles of VRX-007 or vehicle (Lactated Ringer's Solution, USP was used as the vehicle). The only noticeable effects were in the group treated with the highest dose of vector. Three days following injection, all three animals became less active, ceased eating, reduced water intake, and shed fur. At 8-10 days post injection, all animals in this group recovered and remained well until termination of the experiment at 14 days post-injection. No noticeable differences between mock- and vector-injected animals were macroscopically visible upon necropsy. Preliminary results from the histopathological analysis of organs collected form the animals showed no differences between any of the groups. This experiment shows that VRX-007 is tolerated at a dose of at least 1.7×10¹¹ particles following intrajugular injection.

### Example 2

### Syrian Hamster

Infection of Syrian hamster cell lines. An initial experiment was designed to test whether Ad is capable of infecting Syrian hamster cell lines and progressing into late infection. For this experiment, each of four cell lines was infected with VRX-007 or Ad5 at 50 PFU per cell. At 24 h p.i., cells were fixed and immunostained for E1A (an early Ad protein) and DBP (Ad DNA Binding Protein); they were also stained with DAPI (a DNA intercalating dye) to visualize the nuclei. At 48 h p.i., cells were fixed and stained for fiber protein (a late Ad protein that is a component of the capsid) and DBP.

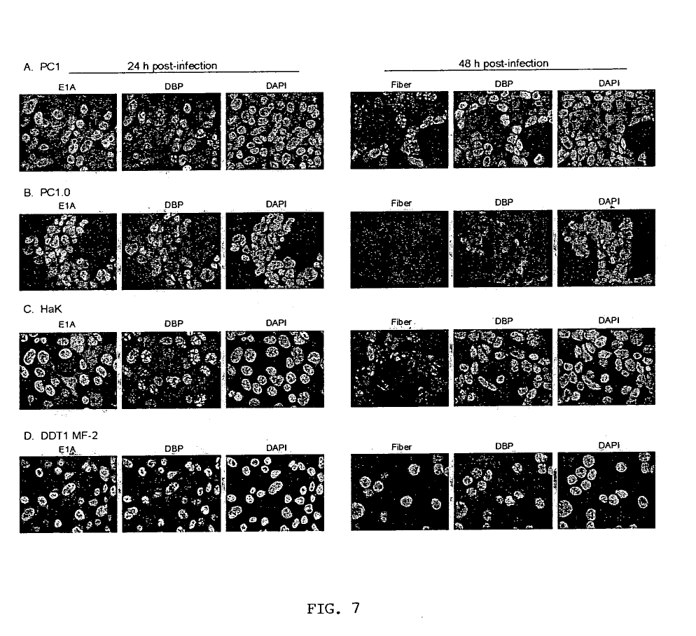

As shown in FIG. 7, the majority of the cells were infected with VRX-007 in each of the four cell lines, as demonstrated by the percentage of cells that were E1A-positive at 24 h p.i. DBP staining was also seen in most of the cells at 24 h p.i. At 48 h p.i., three of the cell lines (PC1, HaK, and DDT1 MF-2) demonstrated fiber staining, indicating progression into late infection (FIG. 7A, C and D). Ad5 infections gave similar results (data not shown). Because progression into late infection requires viral DNA replication, this was the first indication of Ad replication in these cell lines. On the other hand, there was no fiber staining at 48 h for infections of the PC1.0 cell line (FIG. 7B). These experiments suggest that hamster cell lines can be infected with human Ad and that the infection is able to proceed into the late stage in some cell lines.

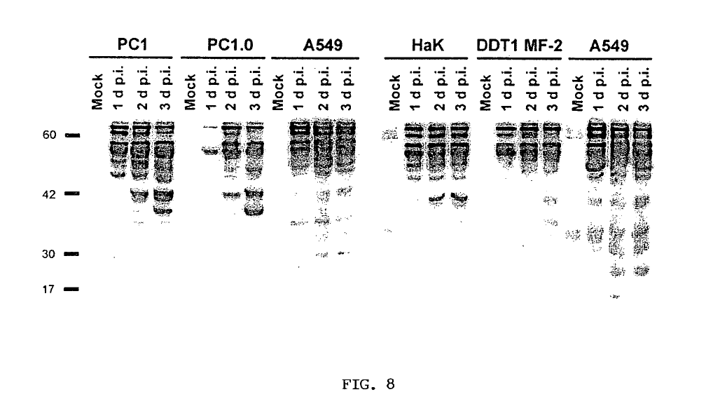

Although the observation that Ad can infect hamster cell lines was interesting, it was not entirely unexpected. Infection merely refers to the uptake of the virus into the cell, and some virus may enter cells at the high MOI that was used. However, replication of Ad implies that the virus infected the cell, the viral nucleocapsid translocated into the cell nucleus, early Ad proteins were expressed which allowed for viral DNA replication, late proteins were expressed, and virions were assembled. To determine if Ad replication entered the late phase in the hamster cell lines, western blot analysis for Ad late proteins was performed on lysates from infected cells. The four hamster cell lines, as well as human A549 cells, were infected with 100 PFU per cell of VRX-007. Cell lysates were prepared at 1, 2 and 3 days post-infection and analyzed by western blotting. The results show that Ad late proteins are expressed in all hamster cell lines suggesting that each cell line is capable of supporting Ad replication (FIG. 8).

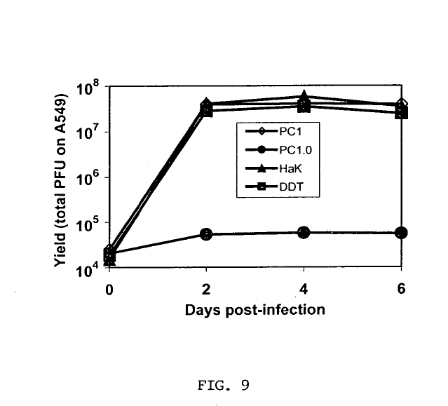

In order to determine the kinetics of replication and the virus yield, a single-step growth analysis was performed for each of the cell lines. This was accomplished by infecting the cells with VRX-007 at 100 PFU per cell to ensure a synchronous infection and, therefore, a single round of infection. At 0, 2, 4, and 6 days p.i., cells were harvested and virus was recovered by freeze-thawing and sonicating the cells. The amount of virus produced at each time point was determined by plaque assay of the crude lysate on A549 cells. As seen in FIG. 9, by 2 days p.i., there was replication yielding more than three logs of virus growth on three of the cell lines tested (PC1, HaK, and DDT1 MF-2). The PC1.0 cell line, however, did not show significant replication, consistent with the lack of fiber by immunostaining. The final yield on the three permissive cell lines was approximately 4×10⁷ PFU. This yield is within an order of magnitude of the yield typically obtained in human A549 cells (data not shown). This experiment demonstrated that VRX-007 is able to replicate quite well in some Syrian hamster cell lines. Replication was observed in these hamster cells despite the deletion of the E3 region of the vector, which is consistent with observations that the E3 region is not required for replication in human cells.

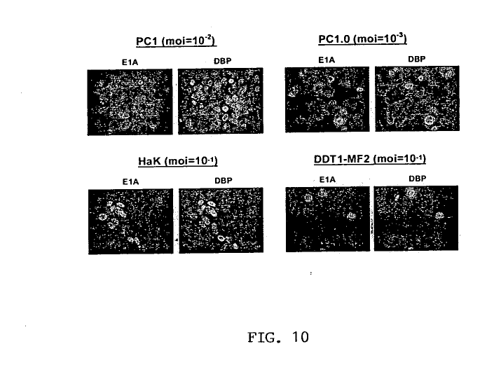

Although the previous experiments established that Ad can infect and replicate quite well in some of these hamster cell lines, in order for this to be an effective model for cancer gene therapy, Ad must be able to spread well from cell to cell through a tumor. A set of experiments to evaluate spread was performed in which spread was visualized by immunofluorescence of Ad proteins. Each of the four cell lines was infected with VRX-007 at serial multiplicities ranging from 10¹ to 10⁻⁴ PFU per cell. At lower multiplicities, only a few cells should be infected initially, and for large foci in the monolayer to demonstrate infection, the virus must have been able to spread from a single infected cell. At 5 days p.i., cells were stained for E1A and DBP to look for areas of adjacent infected cells, demonstrating spread. The results of these spread assays are shown in FIG. 10. Although each of the cell lines illustrated some degree of spread, the PC1 cell line showed the most extensive spread in this assay. This suggests that these cell lines, particularly the PC1 cell line, may potentially be useful in establishing tumors in hamsters as a model for testing VRX-007 and other cancer gene therapy vectors.

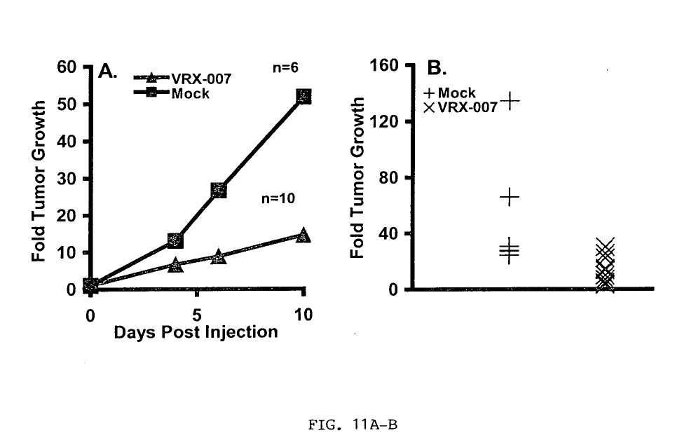

VRX-007 retards the growth of Syrian hamster tumors grown in Syrian hamsters. The Syrian hamster seems to have potential as an animal model for Ad based on our in vitro data. Therefore, the inventors conducted an animal study in which 1×10⁷ DDT1 MF-2 cells were injected subcutaneously into each hind flank of nine Syrian hamsters. Following the appearance of palpable tumors, VRX-007 was injected intratumorally into the tumors of six of the animals. The tumors remaining the other three hamsters were injected with PBS (mock group). Injections were continued for a total of six days, making the total dose of VRX-007 administered 1.8×10⁹ PFU per tumor. Tumor size was measured periodically. In this pilot study, the DDT1 MF-2 tumors provided to be extremely aggressive. By 11 days post-injection, some of the animals had large tumors and, therefore, the animals were sacrificed. The mean tumor growth of the VRX-007 and buffer-injected groups are shown in FIG. 11A. The buffer-injected tumors grew an average of 52-fold (mean volume of 17 ml) while the VRX-007-injected tumors grew only 15-fold (mean volume of 8 ml). Although some of these tumors were somewhat large, the hamsters were observed at least daily and were followed for signs of illness or discomfort. On day four, a hamster in the VRX-007 group had a large tumor that appeared to restrict the use of its leg, so the animal was sacrificed. This hamster had the second largest tumor on day zero and necropsy revealed that the tumor had grown into the muscle. This animal was not used in the final data, as tumor measurements were only available through day four. The fold growth of individual tumors is shown in FIG. 11B. The difference between VRX-007 and buffer treatment groups was determined to be statistically significant by a student's t-test, with a p value of 0.0013. This initial experiment was very promising, especially considering that some of the other hamster cell lines showed equal or even better spread of virus in cell culture.

Necropsy of the animals in this experiment revealed that the tumors of buffer-injected animals were dark-red to brown, semisoft, multilobular, and engorged with blood, some containing large cavities of blood. Many tumors had an area of central necrosis. The tumors from hamsters treated with VRX-007, in addition to being smaller, seemed to have a firmer consistency. The spleens of most of the hamsters appeared to be enlarged. However, the spleen of two hamsters treated with VRX-007 was near normal in size. Another difference observed for the VRX-007 group was the presence of fewer lung lesions. In two of the three animals from the buffer treatment group, multiple raised red lesions were observed in the lungs, while in the six animals treated with VRX-007, three had a single pinpoint raised red lesion. Following necropsy, the organs were preserved by fixation in formalin for histopathological analysis. Histopathological sections will be analyzed to determine if the lung lesions represent metastases from the primary subcutaneous tumor. Although the number of tumors was small and they were quite heterogeneous in size on day zero, there was still a statistically significant suppression of tumor growth by VRX-007. In addition, there was no gross evidence that the virus was dangerous to the animals (the livers will need to be examined histopathologically). This pilot experiment also revealed that the DDT1 MF-2 tumors grew very aggressively.

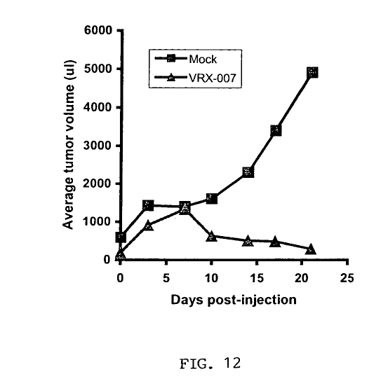

Another pilot animal study was performed using the PC1 tumor cell line. PC1 cells were injected subcutaneously into each flank of each hamster. After tumors developed on one or both sides, they were injected with either vehicle (mock; two tumors) or VRX-007 (2×10¹⁰ PFU/injection; three tumors) on six consecutive days. There were two tumors in the mock group and three tumors in the VRX-007 group. Each of the three tumors injected with VRX-007 reached its maximum size at 3 to 7 days post injection but then began to shrink (FIG. 12). The change in tumor size from day 3 or day 7 to day 21 for the VRX-007-injected tumors was 3,300 μl to 540 μl, 360 μl to 230 μl, and 500 μl to 90 μl. In fact, the last tumor scabbed over and there appeared to be no tumor beneath this scar site. Neither of the tumors in the control group shrank during the course of the experiment.

Yet another study was conducted in Syrian hamsters in which 2×10⁷ HaK cells were injected into each flank of 27 hamsters. Treatment via intratumoral injection began 33 days later when the mean tumor size was about 490 μl. There were 3 treatment groups: buffer (mock), low dose VRX-007 (3×10⁸ PFU per dose), and high dose VRX-007 (1×10¹⁰ PFU per dose). Animals were injected for six consecutive days, for a total initial dose of 1.8×10⁹ PFU (low dose group) or 6×10¹⁰ PFU (high dose group). A second round of intratumoral injections began 18 days after the first set of vector injections. Each group was injected for three consecutive days with buffer, the low dose of VRX-007 (3×10⁸ PFU per dose), or the high dose of VRX-007 (1×10¹⁰ PFU per dose). The total PFU dministered over the course of the experiment was 2.7×10⁹ PFU (low dose group) or 9×10¹⁰ PFU (high dose group).

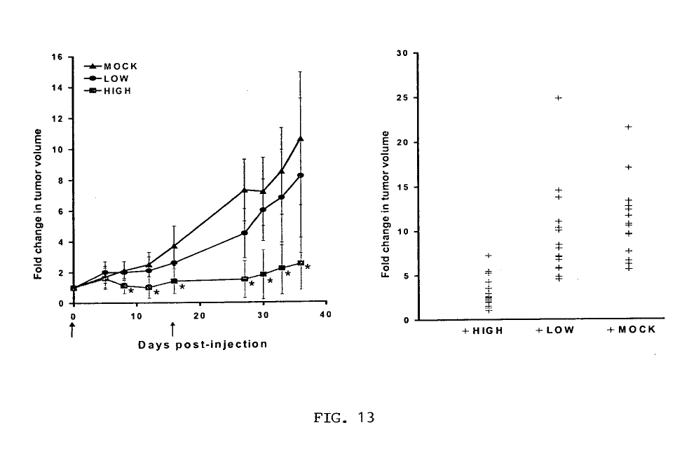

Tumors were measured using a digital caliper and the median fold growth in tumor volume was plotted (FIG. 13). VRX-007 (high dose) was effective in suppressing the growth of HaK tumors as compared to buffer-treated animals. At day 36, the median growth of buffer-treated tumors was 10.6-fold, whereas the median growth of tumors treated with the high dose of VRX-007 was 2.5-fold. Suppression of growth by the high dose of VRX-007 relative to the mock group was statistically significant from the buffer-treated group beginning on day 8, as determined by a Student's t-test. The low dose group was not statistically significantly different from the mock group.

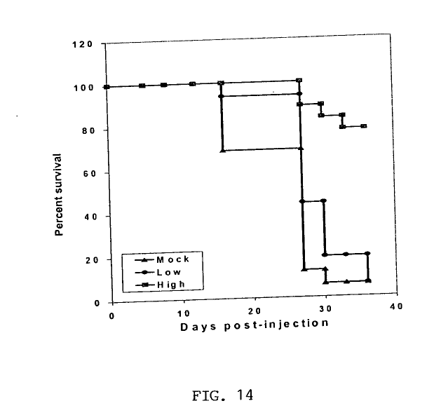

Data from the HaK cell study was also analyzed as a function of survival (FIG. 14). Using SPSS to perform Kaplan-Meier analysis of survival curves, there was a significant difference between the buffer and high dose VRX-007 groups (p<0.0001). At day 36, 14 of 18 (78%) tumors treated with the high dose of VRX-007 did not reach the cutoff volume whereas only one of 16 (6%) of buffer-treated tumors had not yet reached the cutoff volume.

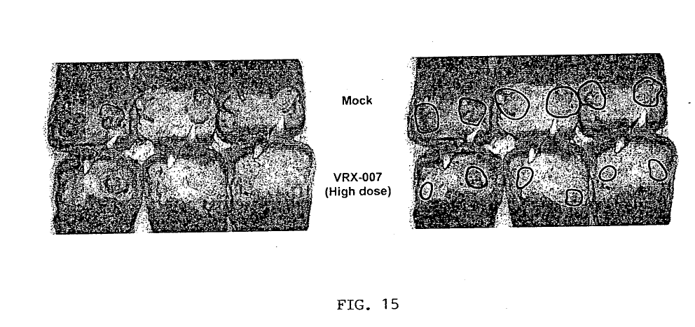

Visually, the tumors that were treated with the high dose of VRX-007 were much smaller than those treated with buffer (FIG. 15). Three animals each from the buffer and high dose VRX-007 treatment groups were photographed at necropsy. Only the hind end of each animal is shown. This end of each animal had been shaved previously so that this area appears as light gray. The animals were aligned so that the hind end of a member of the buffer-treated group (top) faced the hind end of a member of the high dose VRX-007 group (bottom). In addition to the subcutaneous tumor that was treated, some animals in each group had smaller intradermal tumors along the needle track. The right panel of FIG. 15, which is the same photograph as the left panel, shows an outline around the tumor that was injected.

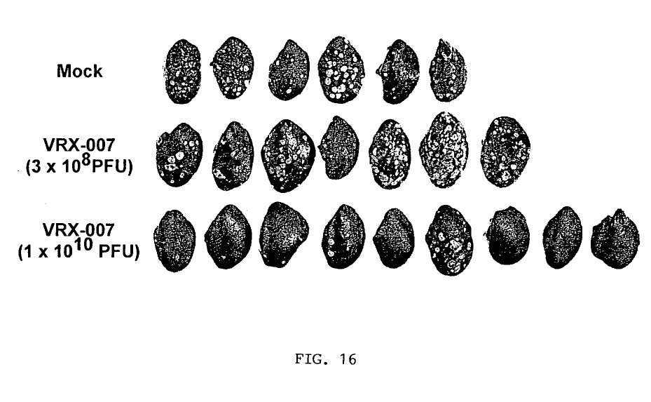

Prior to the end of the study, three buffer-treated and two low dose-treated hamsters had to be sacrificed when these hamsters exhibited severe dyspnea, hunched posture, rough coats, dehydration, and other signs of severe morbidity. Upon necropsy of these hamsters, the inventors observed numerous pulmonary metastases. When additional animals displayed symptoms similar to the aforementioned hamsters, the inventors terminated the study. The inventors compared the degree of metastatic tumor burden in the lungs among groups to determine whether VRX-007 had an effect on the amount of metastasis to the lung. Upon necropsy of the remaining animals, the lungs were stained with India ink to visualize metastases (FIG. 16A). A striking differencewas noticed between the lungs of the buffer-treated and high dose VRX-007-treated animals, the vector-treated animals had far fewer lung metastases and in some cases had no grossly visible metastases. Eight members of the laboratory scored the lungs on an arbitrary scale of zero to five. Samples were blinded according to group and the mean rating for each group was compared (FIG. 16B). The mean score of the lungs of hamsters treated with the high dose of VRX-007 was about 75 percent lower than that of the lungs of the buffer-treated animals.

Acute toxicity in immunocompetent Syrian hamsters following multiple-dose intravenous administration of VRX-007. To determine the maximum tolerated dose (MTD) and examine vector-associated toxicity of VRX-007 using a multiple dosing regimen, Syrian hamsters were intravenously injected with one of three doses of VRX-007 (Table 1). The inventors had determined in a previous study that the MTD of VRX-007 after a single intravenous injection was greater than 1×10¹¹ vector particles (VP). In this experiment, the animals were injected with a daily dose such that the total dose was equivalent to 1 times (low dose), 3 times (medium dose), or 6 times (high dose) the highest dose achieved in the previous study (i.e., the total doses were 1.7×10¹¹ VP, 5.1×10¹¹ VP, and 10^{12×10}¹²vp). Note that the doses in the low and medium dose groups were given in three equivalent doses on three consecutive days while those of the high dose group were divided into two equal parts that were delivered on two consecutive days. A control group received intravenous injection of Lactated Ringer Injection solution, USP on three consecutive days.

TABLE 1
Dose groups for multiple injection MTD/Tox study

| | Daily Dose | | Total Dose | | |
|---|---|---|---|---|---|
| Group | VP^{a} | PFU | VP | PFU | No. of Animals |
| Vehicle | N/A | N/A | N/A | N/A | 5 |
| Low | 5.7 × 10¹⁰ | 6.3 × 10⁹ | 1.7 × 10¹¹ | 1.9 × 10¹⁰ | 6 |
| Medium | 1.7 × 10¹¹ | 1.9 × 10¹⁰ | 5.1 × 10¹¹ | 5.7 × 10¹⁰ | 6 |
| High | 5.1 × 10¹¹ | 5.7 × 10¹⁰ | 1.0 × 10¹² | 1.1 × 10¹¹ | 6 |

^{a}VP = vector particles,
PFU = plaque forming units

Toxicity in the hamsters was assessed by observing the animals for morbidity and mortality, measuring weight gain/loss, determining serum levels of AST, ALT and other clinical chemistry tests, quantifying changes in hematological parameters, and performing gross pathological and histopathological analyses upon sacrifice of the animals. The assessments were conducted according to the schedule shown in Table 2.

Morbidity and Mortality (indicated by [symbol] ) observations. In summary, all hamsters in the high dose group appeared sick after the first injection and died or were sacrificed before day four. Animals in the medium dose group showed some signs of being ill after the second injection, but the animals appeared healthy beyond day 4. However, one animal from this group died seven days after the first injection. All animals in the low dose group appeared healthy throughout the entire experiment. Specific observations are described below (the number in parentheses refer the animal number). Arrrowheads indicate animals that were found dead or that were sacrificed.

Day 1:

High dose: Prior to the second dose on day 1, all hamsters in the high dose were moderately sluggish, ataxic, and had rough coats. When restrained to read the ear punches, they did not resist at all. During the second dose, these hamsters were very quick to succumb to and slow to recover from the anesthetic.

TABLE 2
Schedule of injections and toxicity assessments

| | Day^{a} | | | | | | | | | | | | | | | | |
|---|---|---|---|---|---|---|---|---|---|---|---|---|---|---|---|---|---|
| Item | −4 | −3 | 0 | 1 | 2 | 3 | 4 | 5 | 6 | 7 | 8 | 9 | 10 | 11 | 12 | 13 | 14 |
| Injection | | | X | X | X | | | | | | | | | | | | |
| Morbidity/Mortality | X | X | X | X | X | X | X | X | X | X | X | X | X | X | X | X | X |
| Weight^{b} | | X | X | X | X | X | X | | | X | | | X | | | | X |
| Clinical | X | | | | | | X | | | | | X | | | | | X |
| Chemistry/Hematology^{c} | | | | | | | | | | | | | | | | | |
| Gross | | | | | | | X | | | | | | | | | | X |
| Pathology/Histopathology^{d} | | | | | | | | | | | | | | | | | |

^{a}An “X” indicates that item was assessed on that particular day. Day 0 refers to the first day of injections and days 1, 2, etc. refer to 24, 48, etc. hours post initial injection.
^{b}The weights taken on day −3 were used to randomize the animals by weight prior to the first injection.
^{c}Blood was obtained on the indicated days. Serum levels of AST and ALT were measured for each blood sample. The bleed on day −4 was to establish baseline AST and ALT levels. The complete panel of clinical chemistry tests was performed only on samples collected on days 4 and 14.
^{d}Two animals from the vehicle group and three animals from the low and medium dose groups were sacrificed 4 days after the first injection. The remaining surviving animals were sacrificed 14 days after the first injection. All of the animals from the high dose group died or were sacrificed prior to the first scheduled sacrifice time.

High dose (#17): This animal did not recover from anesthesia following the second dose. The hamster became dyspneic, had infrequent breathing rate and occasional gasps, appeared cyanotic (gray skin appearance around mouth), and became cool to the touch. Approximately 1.5 hours post anesthesia, it became evident that this animal was not going to recover and it was sacrificed.
   Day 2:
High dose: Prior to the third dose, these animals exhibited hunched posture, dyspnea, rough coats, and did not appear to be grooming themselves. They were extremely sluggish, moderately ataxic, and appeared to be cyanotic and dehydrated. Based on these observations and the fact that one animal in this group did not recover from the previous injection, we decided not to administer the final dose to this group. Thus, the high dose group received only two injections for a total of 1.0×10¹² VP.
Medium dose: Prior to the third dose, these animals were slightly sluggish and dehydrated.
High dose (#3 & #15): These two hamsters demonstrated the signs noted earlier in the day for this group and appeared moribund. They were sacrificed at this time.
   Day 3:
High dose (#2, & #24): These two animals were found dead prior to 8 am on day 3. They appeared ill the previous day as described above, but did not seem to be moribund the previous day at the time when two other hamsters in this group were sacrificed.
Medium dose: The animals in this group were less active than vehicle-injected animals. They appeared slightly dehydrated and were not grooming adequately.
High dose (#7): This animal was the only remaining hamster in the high dose group. It exhibited hunched posture and was unresponsive to tapping on the side of the cage. When removed from the cage, the animal was cyanotic, dehydrated, and was not grooming itself. When the animal attempted to walk, it had a very unsteady gait with tremor. This animal was sacrificed at this time.
   Day 4:
Medium dose: These animals were still slightly sluggish.
Scheduled sacrifice of animals (Vehicle #5, & #9; Low dose #16, #21, & #23;
Medium dose #8, #10, & #19)
   Day 7:
Medium dose (#18): This animal was found dead by 8 am.
Medium dose (#4): This hamster was still slightly sluggish and thin.
   Day 14:
Scheduled sacrifice of remaining animals (Vehicle #6, #11, & #20; Low dose #12, #13, & #14; Medium dose #1, & #4)

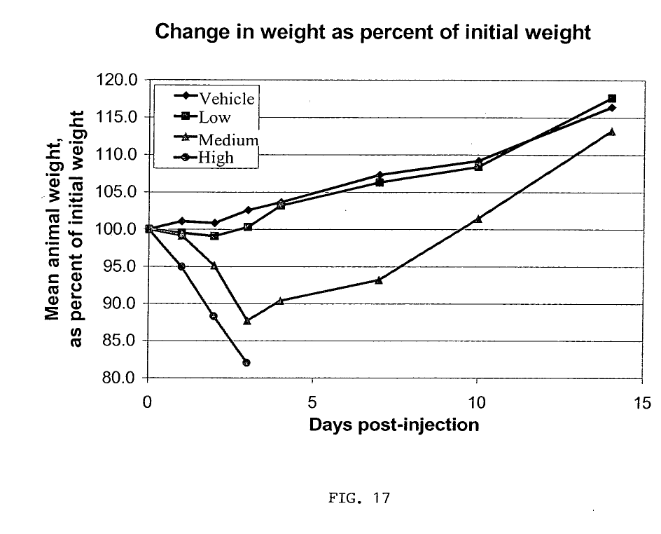

Weight gain/loss measurements. All animals were weighed 3 days prior to the first injection and just before the first injection. Surviving animals were weighed again on days 1 through 4 (inclusive), 7, 10 and 14. The weight of each animal was compared to its weight on day 0 to determine the weight gain or loss for that animal on that day. The mean weight gain or loss was then calculated for each group and the data were plotted as a function of time (FIG. 17). Hamsters in the high dose group exhibited a dramatic and sustained weight loss. Data is only available for days one through three because all of the animals had died or been sacrificed before day four. The medium dose group animals initially showed weight loss, but later gained weight so that by the end of the experiment these animals, on average, gained nearly the same amount of weight as the vehicle control and low dose group animals. The animals in the low dose group showed sustained weight gain equivalent to that shown by the vehicle control group.

Clinical chemistry analyses. Animals in the high dose group that survived long enough to be sacrificed on day two (two animals) or day three (one animal) showed extremely high levels of ALT at the time of sacrifice (Table 3). The level of ALT for animals in the medium dose group showed a wide degree of variation. One animal showed ALT levels throughout the experiment that were comparable to those of the vehicle control group (Table 3). The remaining hamsters in the medium dose group showed high levels of ALT on day 4 (Table 3). By the time of the next bleed (day 9) and continuing to the last bleed (day 13), the levels of ALT returned to near-normal levels for the one remaining animal in this group (the other animals were either sacrificed or had died by this time) (Table 3). Serum ALT levels for the animals in the low dose group were comparable to those in the vehicle control group throughout the course of the experiment (Table 3). Similar results were obtained for serum levels of AST (data not shown).

TABLE 3
Serum levels of ALT at various times.

Day^{b}

| Group^{a} | −4 | 4 | 9 | 14 |
|---|---|---|---|---|
| Vehicle (#9) | 49 | 54 | NA | NA |
| Vehicle (#5) | 49 | 55 | NA | NA |
| Vehicle (#20) | 57 | 42 | 73 | 115 |
| Vehicle (#11) | 54 | 58 | 75 | 61 |
| Vehicle (#6) | 57 | 56 | 54 | 48 |
| Low (#23) | 67 | 79 | NA | NA |
| Low (#16) | 70 | 83 | NA | NA |
| Low (#21) | 66 | 96 | NA | NA |
| Low (#14) | 50 | 160 | 51 | 58 |
| Low (#13) | 50 | 61 | 61 | 67 |
| Low (#12) | 57 | 92 | 55 | 51 |
| Medium (#8) | 44 | 5750 | NA | NA |
| Medium (#10) | 82 | 1106 | NA | NA |
| Medium (#19) | 69 | 2650 | NA | NA |
| Medium (#18) | 42 | 5680 | NA | NA |
| Medium (#4) | 50 | 7150 | 317 | 191 |
| Medium (#1) | 62 | 126 | 58 | 47 |
| High (#2) | 55 | NA | NA | NA |
| High (#15) | 50 | 16150^{c} | NA | NA |
| High (#24) | 52 | NA | NA | NA |
| High (#3) | 62 | 17490^{c} | NA | NA |
| High (#7) | 46 | 8530^{d} | NA | NA |
| High (#17) | 61 | NA | NA | NA |

^{a}The animal number is indicated in parentheses.
^{b}N/A = not applicable because the hamster either died or was sacrificed.
^{c}sacrificed on day 2.
^{d}sacrificed on day 3

Other clinical chemistry assays were performed on the serum samples from days 4 and 14. The data show that total bilirubin (T Bili) and alkaline phosphatase (AP) were elevated in some hamsters in the medium dose group at day 4 (Table 4; elevated bilirubin is indicated by an asterisk), but that the levels of these serum constituents had returned to normal by end of the experiment (14 days post injection) (Table 5).

TABLE 4
Clinical chemistry analyses on day four blood samples.

| | | T Bili | ALP | Creat | BUN | Glu | IP | Ca | TP | Alb |
|---|---|---|---|---|---|---|---|---|---|---|
| Group | ID | (mg/dL) | (U/L) | (mg/dL) | (mg/dL) | (mg/dL) | (mg/dL) | (mg/dL) | (g/dL) | (g/dL) |
| Vehicle | 5 | 0.2 | 235 | 0.5 | 16.0 | 114 | 10.2 | 13.4 | 6.6 | 4.2 |
| Vehicle | 6 | 0.2 | 229 | 0.6 | 15.1 | 100 | 8.8 | 13.3 | 6.7 | 4.0 |
| Vehicle | 9 | 0.3 | 191 | 0.6 | 13.1 | 128 | 10.1 | 13.9 | 6.7 | 4.3 |
| Vehicle | 11 | 0.2 | 256 | 0.4 | | | | | | |
| Vehicle | 20 | 0.2 | 257 | 0.5 | 20.8 | 88 | 9.2 | 13.7 | 6.8 | 4.1 |
| Low | 12 | 0.3 | 290 | 0.6 | 15.0 | 106 | 8.7 | 13.1 | 6.4 | 3.9 |
| Low | 13 | 0.3 | 247 | 0.5 | 15.2 | 100 | 8.8 | 13.2 | 6.5 | 3.8 |
| Low | 14 | 0.1 | 288 | 0.6 | 13.5 | 83 | 6.8 | 12.9 | 5.8 | 3.4 |
| Low | 16 | 0.1 | 262 | 0.6 | 15.6 | 139 | 8.6 | 13.9 | 6.0 | 3.7 |
| Low | 21 | 0.2 | 316 | 0.6 | 14.0 | 136 | 9.1 | 13.9 | 6.1 | 3.7 |
| Low | 23 | 0.1 | 260 | 0.5 | 14.2 | 102 | 9.6 | 12.7 | 5.6 | 3.6 |
| Medium | 1 | 0.1 | 334 | 0.6 | 13.9 | 82 | 8.1 | 11.5 | | |
| Medium | 4* | 7.6 | 954 | | 14.5 | 46 | 5.3 | 11.8 | 5.4 | 3.4 |
| Medium | 8* | 2.2 | 1412 | 0.3 | 14.1 | 48 | 5.4 | 12.8 | 5.0 | 3.0 |
| Medium | 10 | 0.2 | 328 | 0.5 | 12.0 | 86 | 8.3 | 13.2 | 5.4 | 3.1 |
| Medium | 18* | 1.2 | 232 | 1.0 | 42.0 | 40 | 7.0 | 12.5 | 5.2 | 3.1 |
| Medium | 19 | 0.2 | 541 | 0.6 | 4.6 | 71 | 6.6 | 12.2 | 5.4 | 3.2 |

TABLE 5
Clinical chemistry analyses on day fourteen blood samples.

| | | T Bili | AL | Creat | BUN | Glu | IP | Ca | TP | Alb |
|---|---|---|---|---|---|---|---|---|---|---|
| Group | ID | (mg/dL) | (U/L) | (mg/dL) | (mg/dL) | (mg/dL) | (mg/dL) | (mg/dL) | (g/dL) | (g/dL) |
| Vehicle | 6 | 0.1 | 217 | 0.4 | 19.4 | 79 | 9.8 | 13.2 | 6.3 | 4.1 |
| Vehicle | 11 | 0.2 | 164 | 0.4 | 21.1 | 97 | 9.4 | 13.4 | 6.4 | 4.0 |
| Vehicle | 20 | 0.2 | 193 | 0.4 | 22.5 | 106 | 9.1 | 13.8 | 6.5 | 4.0 |
| Low | 12 | 0.3 | 213 | 0.4 | 18.9 | 96 | 8.2 | 13.2 | 6.5 | 3.9 |
| Low | 13 | 0.2 | 180 | 0.5 | 20.2 | 103 | 8.7 | 13.8 | 6.9 | 4.1 |
| Low | 14 | 0.3 | 198 | 0.5 | 20.1 | 95 | 8.9 | 13.5 | 6.8 | 4.0 |
| Medium | 1 | 0.2 | 206 | 0.4 | 19.9 | 101 | 9.9 | 13.1 | 7.2 | 3.7 |
| Medium | 4 | 0.4 | 265 | 0.4 | 14.6 | 102 | 10.0 | 14.0 | 6.8 | 3.8 |

Hematological analyses. Hematological assays were performed on blood samples taken on days four and fourteen. With the exception of platelet counts on day 4 for the animals in the medium dose group, the results from VRX-007-injected hamsters were similar to those of the vehicle-injected animals. The platelet counts at four days post injection from all three animals in the medium dose group were lower than the corresponding counts from the control group and low dose group animals (Table 6). No differences in the hematology results between the vector- and vehicle-injected animals were noted in the day fourteen blood samples (Table 7).

TABLE 6
Hematological analyses on day four blood samples.

| | | WBC | RBC | HGB | HCT | MCV | MCH | MCHC | RDW | PLT |
|---|---|---|---|---|---|---|---|---|---|---|
| Group | ID | (10³/mm³) | (10⁶/mm³) | (g/dl) | (%) | (fl) | (pg) | (%) | (%) | (10³/mm³) |
| Vehicle | 5 | 9.8 | 7.60 | 15.9 | 47.5 | 62 | 20.9 | 33.5 | 13.5 | 743 |
| Vehicle | 9 | 10.9 | 7.79 | 15.8 | 46.8 | 60 | 20.3 | 33.9 | 12.8 | 627 |
| Low | 16 | 12.4 | 6.81 | 14.0 | 42.3 | 62 | 20.6 | 33.2 | 13.6 | 600 |
| Low | 21 | 17.6 | 7.24 | 14.5 | 43.0 | 59 | 20.1 | 33.8 | 13.3 | 603 |
| Low | 23 | 14.1 | 7.21 | 14.9 | 45.0 | 62 | 20.7 | 33.1 | 13.9 | 528 |
| Medium | 8 | 34.8 | 7.41 | 14.9 | 43.5 | 59 | 20.1 | 34.2 | 14.0 | 97 |
| Medium | 10 | 18.7 | 7.06 | 14.4 | 43.0 | 61 | 20.4 | 33.5 | 13.9 | 166 |
| Medium | 19 | 15.5 | 7.68 | 15.8 | 46.3 | 60 | 20.6 | 34.2 | 13.4 | 130 |

| | | MPV | SEG | BAND | LYMPH | MONO | EO | BASO | nRBC |
|---|---|---|---|---|---|---|---|---|---|
| Group | ID | (mm³) | (mm³) | (mm³) | (mm³) | (mm³) | (mm³) | (mm³) | 00 WBC |
| Vehicle | 5 | 6.5 | 1960 | 0 | 7252 | 294 | 294 | 0 | 0 |
| Vehicle | 9 | 6.8 | 1962 | 0 | 8829 | 0 | 109 | 0 | 0 |
| Low | 16 | 6.7 | 2604 | 0 | 8928 | 620 | 248 | 0 | 1 |
| Low | 21 | 6.8 | 2288 | 0 | 14344 | 0 | 704 | 0 | 0 |
| Low | 23 | 7.2 | 2256 | 0 | 10434 | 1128 | 141 | 141 | 0 |
| Medium | 8 | 7.4 | 10788 | 0 | 17748 | 5916 | 348 | 0 | 2 |
| Medium | 10 | 8.1 | 4114 | 0 | 11968 | 2618 | 0 | 0 | 2 |
| Medium | 19 | 6.9 | 5735 | 0 | 8060 | 1395 | 155 | 155 | 1 |

TABLE 7
Hematological analyses on day fourteen blood samples.

| | | WBC | RBC | HGB | HCT | MCV | MCH | MCHC | RDW | PLT |
|---|---|---|---|---|---|---|---|---|---|---|
| Group | ID | (10³/mm³) | (10⁶/mm³) | (g/dl) | (%) | (fl) | (pg) | (%) | (%) | (10³/mm³) |
| Vehicle | 6 | 11.6 | 7.71 | 15.5 | 46.1 | 60 | 20.1 | 33.5 | 13.0 | 714 |
| Vehicle | 11 | 10.0 | 7.69 | 15.5 | 46.9 | 61 | 20.2 | 33.1 | 13.3 | 611 |
| Vehicle | 20 | 12.2 | 7.69 | 15.4 | 46.1 | 60 | 20.0 | 33.4 | 12.6 | 716 |
| Low | 12 | 11.3 | 7.26 | 15.2 | 45.7 | 63 | 21.0 | 33.3 | 13.8 | 927 |
| Low | 13 | 9.6 | 7.51 | 15.1 | 45.5 | 61 | 20.1 | 33.3 | 13.5 | 652 |
| Low | 14 | 11.2 | 7.52 | 15.1 | 45.6 | 61 | 20.1 | 33.1 | 13.5 | 905 |
| Medium | 1 | 11.7 | 6.93 | 14.2 | 42.5 | 61 | 20.5 | 33.5 | 13.8 | 761 |
| Medium | 4 | 5.2 | 7.13 | 14.4 | 43.6 | 61 | 20.1 | 33.0 | 16.9 | 742 |

| | | MPV | SEG | BAND | LYMPH | MONO | EO | BASO | nRBC |
|---|---|---|---|---|---|---|---|---|---|
| Group | ID | (mm³) | (mm³) | (mm³) | (mm³) | (mm³) | (mm³) | (mm³) | 00 WBC |
| Vehicle | 6 | 6.4 | 1624 | 0 | 9512 | 323 | 232 | 0 | 0 |
| Vehicle | 11 | 7.2 | 1200 | 0 | 8400 | 100 | 300 | 0 | 0 |
| Vehicle | 20 | 6.4 | 3904 | 0 | 8052 | 0 | 244 | 0 | 0 |
| Low | 12 | 6.1 | 3729 | 0 | 6554 | 565 | 452 | 0 | 0 |
| Low | 13 | 6.5 | 1248 | 0 | 7584 | 480 | 288 | 0 | 0 |
| Low | 14 | 6.1 | 2408 | 0 | 8232 | 392 | 168 | 0 | 1 |
| Medium | 1 | 6.3 | 3101 | 0 | 8190 | 59 | 351 | 0 | 0 |
| Medium | 4 | 6.9 | 1248 | 0 | 3328 | 364 | 260 | 0 | 0 |

High dose (#17) (Day 1)
The liver had a tan mottled and pitted appearance.
   High dose (#3) (Day 2)
The liver was mottled, pitted, and enlarged. There were dark red foci of congestion.
The colon contained very dark stool.
There was no urine in the bladder.
There was a dark red area in the right lung.
   High dose (#15) (Day 2)
The liver was mottled, pitted, firm and enlarged.
The colon contained very dark stool.
The glandular portion of the stomach and the duodenum were dark red.
The gallbladder was dark red to black and contained no bile.
   High dose (#2) Found Dead (Day 3)
The liver was mottled, pitted, and enlarged.
The gut and the gallbladder were dark red to brown.
The glandular portion of the stomach contained dark spots on the mucosal surface.
The ventral surface of the right median lobe of the lung had a dark hemorrhagic area.
There were emboli in the descending vena cava.
   High dose (#24) Found Dead (Day 3)
The liver was mottled, pitted, and enlarged.
There was dark red to brown stool on the fur of this animal.
There was dark stool in the colon.
There was hemorrhage around the rectum.
The gallbladder was dark red.
The pancreas was edematous.
There was a dark red spot on the left lung.
   High Dose (#7) (Day 3)
Blood was collected for CBC and chemistry panel. A blood smear was done.
There were petecchiae on the skin on the ventral aspect of the animal.
There was very little food in the stomach.
The gallbladder was dark red.
The liver was firm, enlarged, and had tan mottled foci. An impression smear was taken.
The pancreas was edematous and had a necrotic area.
The duodenum was dark red.
The mesenteric lymph nodes were enlarged.
There was dark stool in the colon.
   Vehicle (#5) (Day 4)
No gross pathology noted.
   Vehicle (#9) (Day 4)
No gross pathology noted.
   Low dose (#16) (Day 4)
No gross pathology noted.
   Low dose (#21) (Day 4)
No gross pathology noted.
   Low dose (#23) (Day 4)
No gross pathology noted.
   Medium dose (#8) (Day 4)
The liver was mottled, pitted, had an accentuated lobular pattern and possible fatty change.
   Medium dose (#10) (Day 4)
The liver was mottled, pitted, had an accentuated lobular pattern and possible fatty change.
On the outer surface of the cecum, there were firm white nodules with a dark red surrounding area.
   Medium dose (#19) (Day 4)
The liver was mottled, pitted, had an accentuated lobular pattern and possible fatty change.
On the outer surface of the cecum, there were firm white nodules with a dark red surrounding area.
   Medium dose (#18) Found dead (Day 7)
Reddish-brown urine was noted on the fur of this animal.
The liver was mottled and the gallbladder was markedly enlarged.
   Vehicle (#6) (Day 14)
No gross pathology noted.
   Vehicle (#11) (Day 14)
No gross pathology noted.
   Vehicle (#20) (Day 14
There was a firm nodule near the stomach (possibly a lymph node).
   Low dose (#12) (Day 14)
No gross pathology noted.
   Low dose (#13) (Day 14)
The gallbladder was mildly enlarged.
   Low dose (#14) (Day 14)
No gross pathology noted.
   Medium dose (#1) (Day 14)
The gallbladder was mildly enlarged.
   Medium dose (#4) (Day 14)
The liver was pitted and the gallbladder was thick and mildly enlarged.

Histopathology. Samples of lung, liver, and spleen from each sacrificed animal were preserved by fixation in formalin. These samples were embedded in paraffin and then sectioned and mounted on glass slides. Sections were prepared for histopathological analysis by H&E staining.

All of the tumors and tissues from the first HaK tumor experiment were preserved in formalin for histopathological analysis. With VRX-007, 6 of 18 tumors had areas of necrosis and “degenerating tumor cells.” These necrotic areas were within areas of the viable part of the tumor, not in the central necrotic area that most tumors contained. These necrotic areas had a central area of debris that was primarily cellular (400× dose), immediately surrounded by neutrophil infiltration (400× dose) and more peripherally surrounded by macrophages and fibroblasts. No such areas were seen in the mock-treated tumor.

In a second HaK tumor experiment, the inventor tested not only VRX-007, but also UV-inactivated VRX-007 as a control for the possible immune response against the incoming bolus of virus. The inventors also tested Ad5, a virus named VRX-EGFP, and vehicle. VRX-EGFP has the gene for Enhanced Green Fluorescent Protein inserted downstream from the adp gene in VRX-007. This virus, which expresses large amounts of ADP and EGFP, is an important reagent for studies in the hamster model. HaK cells were injected subcutaneously, and when tumors reached ca. 250 μl they were injected with vehicle, VRX-007, UV-VRX-007, Ad5, or VRX-EGFP. HaK tumors were injected for six consecutive days with 1×10¹⁰ PFU of the indicated vector or virus (the equivalent dose of UV-VRX-007 before inactivation was used) such that the total dose was 6×10¹⁰ PFU.

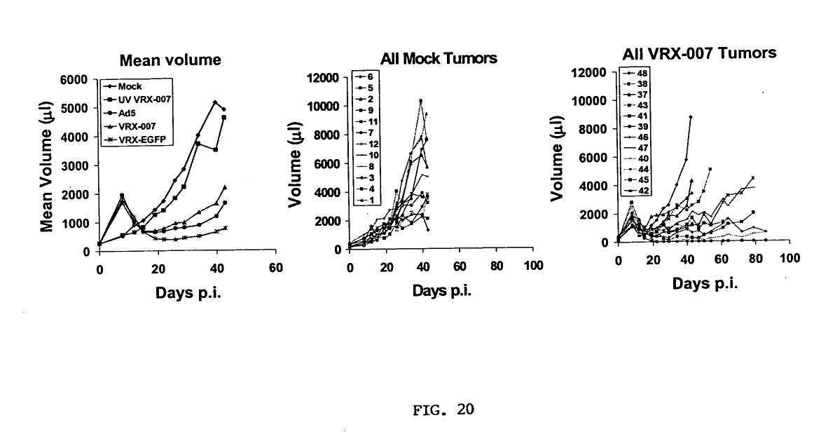

As shown in FIG. 20A, mock- and UV-VRX-007-injected tumors grew steadily to a mean volume of about 5,000 μl by 43 days. In contrast, with VRX-007, Ad5, and VRX-EGFP, there was a remarkable early swelling of the tumors at 8 days, followed by a decline to about 20 days, and then a slow increase in the mean growth until 43 days when this phase of the experiment was stopped because the control tumors had become too large and the animals had to be sacrificed. The VRX-007, Ad5, and VRX-EGFP data were significantly different from the mock and UV-VRX-007 data, but the VRX-007, Ad5, and VRX-EGFP were not statistically different from one another, nor were the mock and UV-VRX-007 different from each other.

In the second phase of this experiment, the inventor allowed the VRX-007-injected tumors to continue to grow. FIG. 20C shows the growth of individual VRX-007-injected tumors over 86 days, and FIG. 13B shows individual mock-injected tumors over 43 days before the animals were sacrificed. Of interest, 1 of 12 VRX-007-injected tumors were completely cured and four were stable. The other tumors grew at different rates, one very fast, three somewhat more slowly, and the others quite slowly.

In summary, the HaK cell line appears to be well suited to study not only subcutaneous tumors but also metastatic cancer. PC1 tumors have the potential to be an orthotopic model. VRX-007 is quite efficacious in suppressing both HaK and PC1 tumors.

When studying Ad vectors in the hamster or cotton rat models, it is important to be able to do immunohistochemistry staining for Ad proteins in various tissues to determine where our Ad vectors replicate. The techniques for immunochemistry have been developed using human Hep3B liver tumors growing in nude mice and injected with the VRX-EGFP virus. EGFP is of course not a virion component so it distinguishes gene expression from the incoming bolus of virus. Also, EGFP is only expressed at late stages of infection by VRX-EGFP, i.e., it was detected in VRX-EGFP-infected cells at 27 h p.i., but not in cells at 27 h that had been treated with AraC to inhibit Ad DNA replication and thereby keep the infection in the early stages; therefore, EGFP expression is a reasonable surrogate for virus replication. The inventor can readily detect the Ad structural proteins, fiber, hexon, and protein pVIII in the same cells expressing EGFP. The ability to detect hexon is important for the general use of the hamster model because the antibody is commercially available and because it reacts with the hexon protein from all serotypes in all subgroups. This would be important for vectors based on other serotypes.

A major advantage of the Syrian Hamster and cotton rat models, as opposed the traditional nude/SCID mouse model, is that interactions between the vector and the host immune system can be examined in these immune competent animals. As shown in FIG. 16, tumor-bearing animals that had been intratumorally injected with 2 doses of 5×10⁹ PFU of VRX-007, VRX-011, VRX-EGFP, or Ad5 and that were bled 14 days post injection were able to mount an anti-Ad antibody response as determined by immunofluorescence (IF) assay using sera obtained at the time of sacrifice. Interestingly, IF staining patterns can be seen for both early and late proteins.

VRX-007 Replicates in HaK Tumors in Syrian Hamsters. The ability of VRX-007 to suppress the growth of tumors is presumed to be due to the ability of the vector to replicate within and to destroy tumor cells. To address whether VRX-007 replicates in HaK tumors, established subcutaneous HaK tumors in Syrian hamsters were injected a single time with 6.9×10⁹ PFU (2.5×10¹¹ virus particles) of VRX-007. As a control, other tumors were injected a single time with 4.0×10′ PFU (2.5×10¹¹ virus particles) of a vector named Ad-EGFP. Ad-EGFP is a replication-defective adenovirus vector that has the adenovirus E1A, E1B, and a portion of the E3 regions deleted. The coding sequences for enhanced green fluorescent protein (EGFP) are inserted into the deleted E1A and E1B regions such that EGFP is expressed. There were three hamsters in the VRX-007 group and three hamsters in the Ad-EGFP group; each hamster had one tumor that was injected. Tumors were harvested at 1.5 hours and 1, 2, 4, 7, and 14 days postinjection. Tumors were disrupted by freezing and thawing, agitation in a TissueLyser, and sonication in phosphate buffered saline (PBS). This procedure extracts the virus into the PBS. The amount of virus in each extract was determined in a tissue culture infection dose 50 (TCID₅₀) assay as described by Condit (2001).

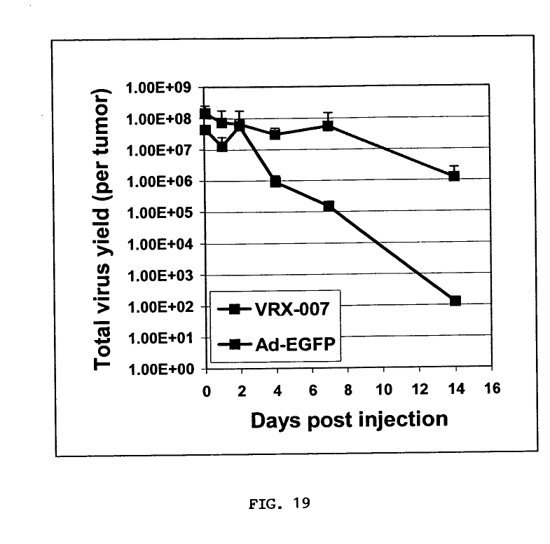

As shown in FIG. 19, more than 10⁸ infection units of VRX-007 were obtained at the 1.5 h time point, and more than 10⁶ units at 14 days. With Ad-EGFP, there were approximately 4 orders of magnitude less infectious virus at 14 days. These data imply that VRX-007 replicates in HaK tumors. Replication of VRX-007 probably explains why VRX-007 is effective at suppressing the growth of HaK tumors in Syrian hamsters.

All of the tumors and tissues from this HaK tumor experiment were preserved in formalin for histopathology examination. Slides for a total of 18 hamsters were prepared, six slides per hamster. These included a slide containing tumor A, tumor B, lung, kidney, liver, or miscellaneous tissues (heart, spleen, pancreas, occasionally lymph node, adrenals or uterus). Tumor A and tumor B refer to the two separate tumors that were formed in the hamster following subcutaneous injection of HaK cells into each hind flank. Only the high-dose and mock-injected (buffer-injected) groups were analyzed. The slides were stained with hematoxylin and eosin (H & E). Except where noted below, no lesions were found in adrenals or uterus.

The tumors examined were all adenocarcinomas. However, there were two morphologic types:
Adenocarcinoma #1: This type of tumor was large, infiltrative, and encapsulated. Cells were arranged in a mixed pattern, primarily papillary, but also solid and glandular. Cells were cuboidal with small, round, hyperchromatic nuclei and scant eosinophilic cytoplasm. Nuclei occasionally contained coarse to clumped chromatin. Occasional nucleoli were prominent and single. Cell borders were distinct. In the papillary pattern, the cells were one to three layers thick on a fibrovascular core. In the glandular pattern, cells were arranged in small irregular glands, generally one to two cells thick. The solid pattern contained sheets of cells. Stroma was moderate and fibrovascular. Mitotic figures were rare <1/400 X. field.
Adenocarcinoma #2: The pattern and arrangement of cells in this type of tumor was similar to Adenocarcinoma #1. However, the cells themselves were more anaplastic. Nuclei were larger, with clumped or marginated chromatin and frequent prominent nucleoli. Cell borders were indistinct. Mitotic figures were 3-4/400 X. field. Stroma was scant. Histologically, this appeared to be a more aggressive tumor.

Several organs contained metastasis of these tumors. Metastases were counted and results were recorded as a range per section of tissue. Frequently, a single metastasis to the lymph node filled the entire node.

What follows is a description of the histopathology observed in the mock and high-dose groups. The numbers in parentheses refer to the animal number in the study.

Mock Animals (#1-9)

MOCK (#1)—Tumors A and B were characterized as adenocarcinoma #1. Metastases: 32-52 lung, 1-2 kidney, two large metastases in the lymph node. No lesions were found in the pancreas, heart, or liver. No spleen was found.

MOCK (#2)—Tumors A and B were characterized as adenocarcinoma #1. Metastases: 39-72, 2-5 kidney. No lesions were found in the liver, spleen, pancreas or heart. No lymph node was found. A large tumor was found on the slide with miscellaneous organs. However, it was unclear if this was a portion of the main tumor, or metastases so extensive that the original organ was completely destroyed.

MOCK (#3)—Tumors A and B were characterized as adenocarcinoma #1. Metastases: 24-43 lung, 0-2 kidney, 1-2 large metastases in the lymph node. No lesions were found in the liver, spleen, heart or pancreas.

MOCK (#4)—Tumors A and B were characterized as adenocarcinoma #1. Metastases: 7-12 lung, 3-5 kidney, one large metastasis in the lymph node. No lesions were found in the liver, spleen, pancreas or heart.

MOCK (#5)—Tumors A and B were characterized as adenocarcinoma #2. However, the mitotic index was slightly lower at 1-3/400X. field. Metastases: 31-34 lung, 0-4 kidney. No lesions were found in liver, spleen, pancreas or heart. No lymph node was found.

MOCK (#6)—Tumors A and B were characterized as adenocarcinoma #1. Metastases: 22-28 lung, 1-2 kidney. No lesions were found in the pancreas, heart, liver or spleen. No lymph node was found.

MOCK (#7)—Tumors A and B were characterized as adenocarcinoma #2. Metastases: 42-65 lung, 5-8 kidney. No lesions were found in the liver, lymph node, spleen, pancreas or heart. Two large tumors were found on the slide with miscellaneous organs. However, it was unclear if these were portions of the main tumors, or metastases so extensive that the original organ was completely destroyed.

MOCK (#8)—Tumors A and B were characterized as adenocarcinoma #2. Metastases: 16-18 lung (many of these were very large), 1-2 mm diameter), 14-17 kidney, two large metastases in the lymph node. A single adenocarcinoma was also noted in the adrenal cortex. However, without special immunostains, I can not distinguish this as a true metastasis versus a spontaneous adenocarcinoma of the adrenal cortex. No lesions were found in the liver pancreas or heart. Examination of the spleen revealed increased granulopoiesis, but not tumor.

MOCK (#9)—Tumors A and B were characterized as adenocarcinoma #2. Metastases: 29-34 lung, 3-5 kidney, one large in the lymph node. No significant lesions were found in the liver, pancreas, heart or spleen.

High Dose VRX-007 Animals (#19-27)

HIGH (#19)—Tumor A—This tumor was variable. Some areas resembled #1, whereas others had the cellular characteristics of #2. Tumor B—This tumor was similar to A. However, much of the tumor was necrotic and many of the tumor cells were degenerating. For both tumors mitotic index was <1/400 X. field. Metastases: 3-12 lung. No lesions were found in the liver, kidney, spleen, heart, or pancreas. No lymph node was found.

HIGH (#20)—Tumor A and B—These tumors were variable. Some areas resembled #1, whereas others had the cellular characteristics of #2. However, many of the tumor cells were degenerating with scattered foci of apoptosis and large necrotic areas. Metastases: 3-22 (very small, approximately 1/4 the size of metastases previously reported) lung. No lesions were found in the kidney, liver, pancreas heart or spleen. No lymph node was found.

HIGH (#21)—Tumors A and B were characterized as adenocarcinoma #1. Metastases: 2-7 lung, 0-1 kidney, one large metastasis lymph node. No lesions were found in the liver, pancreas, spleen or heart. A large tumor was found on the slide with miscellaneous organs. However, it was unclear if this was a portion of the main tumor, or metastases so extensive that the original organ was completely destroyed.

HIGH (#22)—Tumors A and B were characterized as adenocarcinoma #1. Metastases: 6-15 lung, 1-4 kidney. No significant lesions were found in the liver, pancreas, lymph node or heart. No spleen was found.

HIGH (#23)—Tumors A and B were characterized as adenocarcinoma #2. However, mitotic index was lower at 0-3/400 X. field, and there were frequent degenerating cells and foci of apoptosis. No lesions were found in the lung, liver, kidney, pancreas, spleen or heart. A large tumor was found on the slide with miscellaneous organs. This tumor contained a rim of lymphocytes beneath a capsule suggesting that it may have been extensive infiltration of a lymph node.

HIGH (#24)—Tumors A and B were characterized as adenocarcinoma #1. Metastases: 24-28 lung, 0-2 kidney. No lesions were found in the liver, pancreas, heart or spleen. No lymph node was identified.

HIGH (#25)—Tumors A and B were characterized as adenocarcinoma #1. Metastases: 01—lung. No lesions were found in the liver, kidney, pancreas or heart. No spleen or lymph node was found.

HIGH (#26)—Tumor A—This tumor was variable. Some areas resembled #1, whereas others had the cellular characteristics of #2. However, many of the tumor cells were degenerating with scattered foci of apoptosis and large necrotic areas. Tumor B was characterized as adenocarcinoma #2. However, mitotic index was lower at 0-3/400X. field. No lesions were found in the lung, liver, kidney, pancreas, spleen or heart. No lymph node was found.

HIGH (#27)—Tumors A and B were characterized as adenocarcinoma #2. Metastases: 0-1 lung, 0-1 lymph node (large metastasis). No lesions were found in the kidney, heart, liver, spleen or pancreas.

The following is a description of the histopathology analysis. The numbers refer to the animal numbers prior to randomization. The day noted refers to the date the animal was sacrificed or found dead. “FD” refers to an animal that was found dead. Animal #18 had moderate to severe post mortem degeneration that limited the evaluation. A summary of the analysis is provided at the end of this description.

Liver

High Dose:

#17/Day 1—Examination of the liver revealed multifocal, moderate necrosis with minimal influx of neutrophils. Occasional hepatocytes with intranuclear inclusions were noted.

#3/Day 2—Examination of liver revealed massive, diffuse hemorrhage and necrosis. Many hepatocytes contained intranuclear inclusions.

#15/Day 2—Examination of liver revealed severe, diffuse hemorrhage and necrosis. Numerous hepatocytes contained intranuclear inclusions.

#2/Day 3 (**FD**)—Examination liver revealed severe, diffuse, multifocal hemorrhage and necrosis. Several intranuclear inclusions were noted in hepatocytes.

#24/Day 3 (**FD**)—Examination of liver revealed severe, diffuse necrosis and hemorrhage with scattered hepatocytes containing intranuclear inclusions.

#7/Day 3—Examination of the liver revealed severe, diffuse necrosis and hemorrhage, with numerous intranuclear inclusions within hepatocytes.

Intermediate Dose:

#8/Day 4—Examination of the liver revealed severe, multifocal coagulative necrosis. The pattern was random. A few hepatocytes contained intranuclear inclusions.

#10/Day 4—Examination of the liver revealed numerous, scattered mitotic figures and scattered apoptotic hepatocytes. There was a small focal accumulation of macrophages and fibroblasts at the periphery of the liver.

#19/Day 4—Examination of the liver revealed moderate, multifocal coagulative necrosis with minimal neutrophilic infiltrates.

#18/Day 7 (**FD**)—Examination liver revealed severe, multifocal hemorrhage and necrosis.

#1/Day 14—A single large focus of hemorrhage was noted in the liver.

#4/Day 14—Examination of the liver revealed no significant lesions. There was distention of hepatocytes as with glycogen and scattered foci of extramedullary hematopoiesis, but these are normal incidental findings.

Mock:

#5/Day 4—Examination of the liver revealed scattered hepatocyte nuclei which appeared hyperchromatic or pyknotic.

No Significant Lesions:

Low dose (#16)/Day 4
Low dose (#21)/Day 4
Low dose (#23)/Day 4
Low dose (#12)/Day 14
Low dose (#13)/Day 14
Low dose (#14)/Day 14
Mock (#9)/Day 4
Mock (#6)/Day 14
Mock (#11)/Day 14
Mock (#20)/Day 14
   Spleen

High dose:

#17/Day 1—Examination the spleen revealed lymphoid depletion with an influx of polymorphonuclear cells into the lymphoid follicles.

#3/Day 2—Examination the spleen revealed increased macrophage numbers and decreased erythrocytic precursors. There was severe lymphoid depletion with increased apoptotic figures.

#15/Day 2—Examination the spleen revealed severe congestion, and lymphoid depletion with scattered apoptotic cells within the lymphoid follicles. There was also decreased erythropoesis and granulopoiesis.

#2/Day 3 (**FD**)—Examination of the spleen revealed increased numbers of macrophages, increased apoptotic foci within lymphoid follicles, and decreased erythrocytic precursors.

#24/Day 3 (**FD**)—Examination of the spleen revealed severe congestion, lymphoid depletion with many degenerate/necrotic lymphocytes.

#7/Day 3—Examination the spleen revealed numerous apoptotic figures within the lymphoid follicles. There were increased numbers of immature lymphocytes. Increased numbers of macrophages and granulocytes were also noted. There was a decrease in numbers of erythrocytic precursors.

Intermediate dose:

#8/Day 4—Examination of the spleen revealed mild lymphoid hyperplasia, increased granulopoiesis, increased numbers of macrophages and decreased erythropoesis.

#10/Day 4—Examination of the spleen revealed mild lymphoid hyperplasia, and decreased erythropoesis.

#19/Day 4—Examination of the spleen revealed increased granulopoiesis, increased macrophage numbers and decreased erythropoesis.

#18/Day 7 (**FD**)—Examination of the spleen revealed severe lymphoid depletion, increased numbers of plasma cells and macrophages, and decreased erythropoesis.

Low dose:

#12/Day 14—There was mild lymphoid hyperplasia in the spleen.

#13/Day 14—Examination the spleen revealed mild lymphoid hyperplasia.

#14/Day 14—There was mild lymphoid hyperplasia in the spleen.

Mock:

#5/Day 4—Examination of a small section of spleen revealed marked lymphoid depletion. Lymphoid follicles were reduced in size and number. There was also a decrease in the number of erythrocytic precursors.

No Significant Lesions:

Intermediate dose (#1)/Day 14
Intermediate dose (#4)/Day 14
Low dose (#16)/Day 4
Low dose (#21)/Day 4
Low dose (#23)/Day 4
Mock (#9)/Day 4
Mock (#11)/Day 14
Mock (#20)/Day 14
   Pancreas

High dose:

#3/Day 2—Examination of the pancreas revealed moderate, interstitial edema and scattered apoptotic figures within the exocrine acini. There was also multifocal degeneration and necrosis of the islet cells.

#15/Day 2—Examination of the pancreas revealed moderate interstitial edema, scattered apoptotic cells within acini and degeneration and vacuolation of islet cells.

#2/Day 3 (**FD**)—In the pancreas, there was minimal to mild interstitial edema.

#24/Day 3 (**FD**)—Examination of pancreas revealed interstitial edema, with a few foci of degeneration and necrosis within exocrine acini.

#7/Day 3—Examination of the pancreas revealed mild interstitial edema and minimal interstitial hemorrhage.

Intermediate dose:

#18/Day 7 (**FD**)—Examination the pancreas revealed mild interstitial edema and degeneration of islet cells.

No Significant Lesions:

High dose (#17)/Day 1
Intermediate dose (#8)/Day 4
Intermediate dose (#10)/Day 4
Intermediate dose (#1)/Day 14
Intermediate dose (#4)/Day 14
Low dose (#16)/Day 4
Low dose (#21)/Day 4
Low dose (#12)/Day 14
Low dose (#13)/Day 14
Low dose (#14)/Day 14
Mock (#5)/Day 4
Mock (#9)/Day 4
Mock (#6)/Day 14
Mock (#11)/Day 14
Mock (#20)/Day 14
   GI Tract

High dose:

#3/Day 2—Examination of the cecum revealed degeneration and necrosis of the crypt mucosa, and hemorrhage into the lumen.

#15/Day 2—Examination of the stomach revealed focal congestion and degeneration of mucosal cells. There were also scattered foci of spherical, pigmented material which could not be identified.

#15/Day 2—Examination of the intestine revealed severe diffuse congestion, hemorrhage and necrosis of the mucosa.

#2/Day 3 (**FD**)—Examination of the intestine revealed severe, diffuse necrosis of apical portions of crypts. The lumen of the intestine was filled with blood.

#2/Day 3 (**FD**)—Examination of the stomach revealed moderate, diffuse degeneration and necrosis of the mucosal epithelium. Many of the epithelial cells contained intranuclear inclusions.

#2/Day 3 (**FD**)—Examination of cecum/colon revealed mild to severe, mucosal necrosis and ulceration with massive hemorrhage into the lumen.

#2/Day 3 (**FD**)—There was moderate, segmental necrosis of the mucosa in the small bowel.

#24/Day 3 (**FD**)—Examination the small intestine revealed mild congestion within the lamina propria.

#7/Day 3—An abscess was noted within the mesentery.

#7/Day 3—There was moderate hyperplasia of the mesenteric lymph nodes.

#7/Day 3—In the small bowel, there was focal congestion within the lamina propria.

Intermediate dose:

#18/Day 7 (**FD**)—Post mortem degeneration was too severe to evaluate the intestine.

Mock:

#20/Day 14—A well encapsulated abscess was found in the mesentery near the pancreas.

NO SIGNIFICANT LESIONS (only animals with gross GI findings were submitted):

High dose (#15)/Day 2
High dose (#7)/Day 3
Intermediate dose (#10)/Day 4
   Lung

High Dose:

#3/Day 2—Examination of the lung (second slide) revealed mild congestion and hemorrhage with increased numbers of alveolar macrophages.

#2/Day 3 (**FD**)—Examination of the lung (second slide) revealed severe, diffuse perivascular hemorrhage, with mild to moderate diffuse alveolar hemorrhage.

Low Dose:

#13/Day 14—Examination of the lung revealed a small granuloma surrounding foreign material (aspiration). The remainder of the lung was normal.

No Significant Lesions:

High dose (#17)/Day 1
High dose (#15)/Day 2
High dose (#24)/Day 3 (**FD**)
High dose (#7)/Day 3
Intermediate dose (#8)/Day 4
Intermediate dose (#10)/Day 4
Intermediate dose (#19)/Day 4
Intermediate dose (#18)/Day 7 (**FD**)
Intermediate dose (#1)/Day 14
Intermediate dose (#4)/Day 14
Low dose (#16)/Day 4
Low dose (#21)/Day 4
Low dose (#23)/Day 4
Low dose (#12)/Day 14
Low dose (#14)/Day 14
Mock (#5)/Day 4
Mock (#9)/Day 4
Mock (#6)/Day 14
Mock (#11)/Day 14
Mock (#20)/Day 14
   Kidney

High Dose:

#3/Day 2—There was minimum congestion of the glomeruli of the kidney.

Low Dose:

#12/Day 14—Examination the kidney revealed mild diffuse congestion.

Mock:

#11/Day 14—Mild diffuse congestion was noted in the kidney.

No Significant Lesions:

High dose (#17)/Day 1
High dose (#15)/Day 2
High dose (#2)/Day 3 (**FD**)
High dose (#24)/Day 3 (**FD**)
High dose (#7)/Day 3
Intermediate dose (#8)/Day 4
Intermediate dose (#10)/Day 4
Intermediate dose (#19)/Day 4
Intermediate dose (#18)/Day 7 (**FD**)
Intermediate dose (#1)/Day 14
Intermediate dose (#4)/Day 14
Low dose (#16)/Day 4
Low dose (#21)/Day 4
Low dose (#23)/Day 4
Low dose (#14)/Day 14
Mock (#5)/Day 4
Mock (#9)/Day 4
Mock (#6)/Day 14
Mock (#20)/Day 14
   Heart
   No Significant Lesions:

All animals.

Summary of histopathology analysis. Evaluation of tissues from animals that received the low dose of VRX-007 revealed no consistent significant findings. The intermediate dose (excluding the animal that died on day 7) was associated with hepatocyte necrosis at day 4 but not day 14. The animal that died on day 7 in the intermediate dose group had findings in the liver, spleen, and pancreas that resembled those found in the high dose group. The high dose group showed multiple abnormalities including hemorrhage, necrosis, and intranuclear inclusions in the liver. In the stomach, small, and large intestines, there was necrosis of the mucosa with hemorrhage into the lumen. There was also hemorrhage in the lungs of 2 out of 6 animals. Lymphoid depletion and increased apoptotic figures were seen within the lymphoid follicles in the spleen. Examination of the pancreas revealed interstitial edema, apoptotic cells in the exocrine acini, and necrosis of islet cells.

Summary of the MTD Study in Syrian Hamsters. The inventors have developed the Syrian hamster as a permissive immunocompetent model to evaluate the safety and efficacy of replication-competent adenovirus vectors for cancer gene therapy. One of these vectors is named VRX-007; VRX-007 overexpresses ADP, an Ad protein that mediates virus release and cell-to-cell spread. In order to determine the maximum tolerated dose (MTD) of VRX-007 in hamsters, they administered a single injection of VRX-007 into the jugular vein. Hamsters received 1.9×10⁸ PFU, 1.9×10⁹ PFU, or 1.9×10¹⁰ PFU. No morbidity and no gross or histopathologic abnormalities were observed. In a second study, they administered VRX-007 via the jugular vein on three consecutive days. The cumulative doses were 1.9×10¹⁰ PFU for the low dose group, 5.7×10¹⁰ PFU for the intermediate dose group, and 1.1×10¹¹ PFU for the high dose group. Half of each group was sacrificed on day 4, and the remainder of the animals were sacrificed on day 14. Hamsters in the low dose group did not display signs of morbidity, and no significant findings were observed grossly at either sacrifice date. In the intermediate group, one animal died on day 7; at necropsy, there was mild to moderate gross liver pathology at day 4 but only mild liver pathology at day 14. All animals in the high dose group were either found dead or became moribund and were sacrificed by three days postinjection. Liver enzymes (ALT and AST) in the serum were measured in order to evaluate the degree of hepatotoxicity. These enzymes were normal in the low dose group. For the intermediate group, the ALT and AST levels generally correlated with the degree of gross liver pathology observed at necropsy, with mild to moderate elevation at 4 days that resolved at 14 days. In the histopathology analysis, little toxicity was observed in the low dose group, moderate mostly liver toxicity was seen in the intermediate group on day 4 that resolved on day 14 (with the exception of the animal that died), and extensive toxicity was obtained in the high dose group. For the low and intermediate groups, elevation of alkaline phosphatase levels as well as a decrease in total protein and albumin levels were observed at 4 but not 14 days. For both groups, hematological analysis revealed a dose-dependent decrease in platelet counts on day four, although platelet counts were normal again at 14 days. Based on these data, the inventors estimate the MTD of VRX-007 when administered intravenously on three consecutive days into Syrian hamsters to be the low dose used in this study (1.9×10¹⁰ PFU total or 1.7×10¹¹ VP total). This corresponds to a dose of 2.1×10¹¹ PFU/kg (1.9×10¹² VP/kg).

All of the methods and compositions disclosed and claimed herein can be made and executed without undue experimentation in light of the present disclosure. While the compositions and methods of this invention have been described in terms of preferred embodiments, it will be apparent to those of skill in the art that variations may be applied to the methods and compositions and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,415,732
U.S. Pat. No. 4,458,066
U.S. Pat. No. 4,578,770
U.S. Pat. No. 4,596,792
U.S. Pat. No. 4,599,230
U.S. Pat. No. 4,599,231
U.S. Pat. No. 4,601,903
U.S. Pat. No. 4,608,251
U.S. Pat. No. 5,354,855
U.S. Pat. No. 5,670,488
U.S. Pat. No. 5,677,178
U.S. Pat. No. 5,747,869
U.S. Pat. No. 5,795,715
U.S. Pat. No. 5,795,715
U.S. Pat. No. 5,889,136
U.S. Pat. No. 5,981,225
U.S. Pat. No. 6,069,134
U.S. Pat. No. 6,136,594
U.S. Pat. No. 6,143,290
U.S. Pat. No. 6,410,010
U.S. Pat. No. 6,511,184
U.S. Pat. No. 6,627,190
Bosher et al., Nat. Cell. Biol., 2(2):E31-E36, 2000.
Caplen et al., Gene, 252(1-2):95-105, 2000.
Condit, R. C., Principles of Virology. In Fields Virology, Knipe, D. M. and Howley P. D., eds., vol. 1, p.p. 19-51, 2001.
Cook et al., Cell, 27:487-496, 1981.
Couch et al., Am. Rev. Resp. Dis., 88:394-403, 1963.
Egami et al., Am. J. Pathol., 138:557-561, 1991.
Elbashir et al., Genes Dev., 5(2):188-200, 2001.
Elbashir et al., Nature, 411(6836):494-498, 2001.
Fire et al., Nature, 391(6669):806-811, 1998.
Forster and Symons, Cell, 49(2):211-220, 1987.
Gerlach et al., Nature (London), 328:802-805, 1987.
Gomez-Foix et al., J. Biol. Chem., 267:25129-25134, 1992.
Graham and Prevec, Biotechnology, 20:363-390, 1992.
Graham et al, J. General Virology, 36:59-74, 1977.
Grishok et al., Science, 287:2494-2497, 2000.
Grunhaus and Horwitz, Seminar in Virology, 3:237-252, 1992.
Herz and Gerard, Proc. Natl. Acad. Sci. USA, 90:2812-2816, 1993.
Hjorth et al., Arch. Virol., 100:279-283, 1988
Joyce, Nature, 338:217-244, 1989.
Ketting et al., Cell, 99(2):133-141, 1999.
Khoobyarian et al., J. Surg. Oncol., 7:421-425, 1975.
Kim and Cook, Proc. Natl. Acad. Sci. USA, 84(24):8788-8792, 1987.
Le Gal La Salle et al., Science, 259:988-990, 1993.
Levrero et al., Gene, 101:195-202, 1991.
Lin et al., Genetics, 153:1245-1256, 1999.
Michel and Westhof, J. Mol. Biol., 216:585-610, 1990.
Montgomery et al., Proc. Natl. Acad. Sci. USA, 95:15502-15507, 1998.
Morin et al., Proc. Natl. Acad. Sci. USA, 84:4626-4630, 1987.
Pacini et al., J. Infect. Dis., 150:92-97, 1984.
PCT Appln. WO 00/44914
PCT Appln. WO 01/68836
PCT Appln. WO 99/32619
PCT Appln. WO 01/36646
Prince et al., J. Virol., 67:101-111, 1993.
Racher et al., Biotechnology Techniques, 9:169-174, 1995.
Ragot et al., Nature, 361:647-650, 1993.
Reinhold-Hurek and Shub, Nature, 357:173-176, 1992.
Remington's Pharmaceutical Sciences” 15^{th} Edition, pages 1035-1038 and 1570-1580, 1980.
Renan, Radiother. Oncol., 19:197-218, 1990.
Rich et al., Hum. Gene Ther., 4:461-476, 1993.
Rosenfeld et al., Science, 252:431-434, 1991.
Rosenfeld, et al., Cell, 68:143-155, 1992.
Sarver et al., Science, 247:1222-1225, 1990.
Scanlon et al., Proc. Natl. Acad. Sci. USA, 88:10591-10595, 1991.
Sharp et al., Science, 287:2431-2433, 2000.
Sharp, Genes Dev., 13:139-141, 1999.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Tabara et al., Cell, 99(2):123-132, 1999.
Top et al., J. Infect. Dis., 124:155-160, 1971.
Wagner et al., Science, 260:1510-1513, 1993.
Wilder et al., Cancer Res., 59:410-413, 1999b.
Wilder et al., Gene Therapy, 6:57-62, 1999a.
Wildner and Morris, Hum. Gene Ther., 13:101-112, 2002.
Wincott et al., Nucleic Acids Res., 23(14):2677-2684, 1995.
Oualikene et al., J. Gen. Virol. 75, 2765-2768, 1994.
Rojas-Martinez et al., Cancer Gene Ther. 5, 365-370, 1998.
Oualikene et al., J. Gen. Virol. 75, 2765-2768, 1994.
Rojas-Martinez et al., Cancer Gene Ther. 5, 365-370, 1998.
Wildner, O. and Morris, J.C., Hum. Gene Ther. 13, 101-112, 2002.

1. A method of evaluating an adenoviral vector for anti-tumor effects comprising:
(a) providing a Syrian hamster that comprises a hamster cancer cell;
(b) administering to said hamster a recombinantly-engineered adenoviral vector; and
(c) assessing the effect of said adenoviral vector on said cancer cell.

2. The method of claim 1, wherein said adenoviral vector is replication competent.

3. The method of claim 1, wherein said adenoviral vector overexpresses ADP relative to wild-type adenovirus serotype 5.

4. The method of claim 1, wherein said adenoviral vector is replication deficient.

5. The method of claim 1, wherein said adenoviral vector is VRX-001, VRX-002, VRX-003, VRX-004, VRX-005, VRX-006, VRX-007, VRX-008, VRX-009, VRX-010, VRX-011, VRX-012, VRX-013, VRX-014, VRX-015, VRX-016, VRX-017, VRX-018, VRX-019, VRX-020, VRX-021, INGN:201, INGN:241, or INGN:251.

6. The method of claim 1, wherein said adenoviral vector comprises a heterologous coding sequence.

7. The method of claim 6, wherein said heterologous coding sequence comprises a tumor suppressor, an inducer of apoptosis, a cell cycle regulator, a toxin, an enzyme, a hormone, a cytokine, an antisense DNA or RNA directed against an oncogene, an siRNA, a single-chain antibody, an inhibitor of angiogenesis, a metalloprotease inhibitor or a peptide hormone.

8. The method of claim 7, wherein said tumor suppressor is p53, mda-7, Rb, p16, or PTEN.

9. The method of claim 7, wherein said inducer of apoptosis is Bax, Bad, Bik, Bid, or Bcl-Xₛ.

10. The method of claim 7, wherein said cell cycle regulator is p300/CBP.

11. The method of claim 7, wherein said toxin is pertussis toxin or ricin.

12. The method of claim 7, wherein said enzyme is HSV-tk.

13. The method of claim 7, wherein said hormone is a LHRH analog, estrogen, progestin or an anti-androgen.

14. The method of claim 7, wherein said antisense DNA or RNA is directed to ras, raf myb, myc, or src.

15. The method of claim 7, wherein said cytokine is tumor necrosis factor alpha, Fas ligand, tumor necrosis factor-related apoptosis-inducing ligand (TRAIL),IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-24, interferon-α, interferon-β, and interferon-γ.

16. The method of claim 7, wherein said siRNA is directed to ras, raf, myb, myc, or src.

17. The method of claim 7, wherein said single-chain antibody is directed to ras, raf myb, myc, or src.

18. The method of claim 1, wherein said adenoviral vector lacks one or more regions selected from the E1, E2, E3, E4, protein pIX, protein IVₐ₂, and major late transcription units.

19. The method of claim 1, wherein said hamster cancer cell is comprised within a solid tumor.

20. The method of claim 1, wherein said hamster cancer cell is comprised within a metastatic lesion.

21. The method of claim 1, wherein said hamster cancer cell is a pancreatic carcinoma cell, a leiomyosarcoma cell, or a kidney tumor-forming cell.

22. The method of claim 1, wherein administering comprises intratumoral or intralesional injection.

23. The method of claim 1, wherein admininstering comprises local, regional or systemic administration.

24. The method of claim 1, wherein administering comprises subcutaneous, intranasal, intramuscular, intravenous, intra-arterial, intraperitoneal or oral administration.

25. The method of claim 19, wherein assessing comprises measuring a change in tumor volume or diameter following treatment with said adenoviral vector.

26. The method of claim 20, wherein assessing comprises measuring metastatic growth following treatment with said adenoviral vector.

27. The method of claim 1, wherein assessing comprises measuring development of metastases following treatment with said adenoviral vector.

28. The method of claim 1, wherein assessing comprises measuring tumor cell apoptosis following treatment with said adenoviral vector.

29. The method of claim 19, wherein assessing comprises measuring tumor necrosis following treatment with said adenoviral vector.

30. The method of claim 1, wherein assessing comprises measuring tumor infiltration of adjacent tissues following treatment with said adenoviral vector.

31. The method of claim 1, wherein assessing comprises measuring production of a tumor-derived compound following treatment with said adenoviral vector.

32. The method of claim 1, wherein assessing comprises measuring adenoviral-based toxicity in said hamster.

33. The method of claim 1, wherein assessing comprises measuring adenoviral-based death of said hamster.

34. The method of claim 1, wherein said adenoviral vector is administered more than once.

35. The method of claim 33, wherein assessing is performed once at the conclusion of all administrations.

36. The method of claim 33, wherein assessing is performed between at least a first and a second administration.

37. The method of claim 1, further comprising administering a second non-adenoviral therapy to said hamster, and assessing the combined effect of said adenoviral vector and said second non-adenoviral therapy.

38. A method of evaluating an adenoviral vector for anti-tumor effects comprising:
(a) providing a cotton rat that comprises a cotton rat cancer cell;
(b) administering to said cotton rat an adenoviral vector; and
(c) assessing the effect of said adenovirus on said cancer cell.

39. The method of claim 38, wherein said adenoviral vector is a recombinantly-engineered adenoviral vector.

40. The method of claim 38, wherein said adenoviral vector is replication competent.

41. The method of claim 38, wherein said adenoviral vector overexpresses ADP relative to wild-type adenovirus serotype 5.

42. The method of claim 38, wherein said adenoviral vector is replication deficient.

43. The method of claim 38, wherein said adenoviral vector is VRX-001, VRX-002, VRX-003, VRX-004, VRX-005, VRX-006, VRX-007, VRX-008, VRX-009, VRX-010, VRX-011, VRX-012, VRX-013, VRX-014, VRX-015, VRX-016, VRX-017, VRX-018, VRX-019, VRX-020, VRX-021, INGN:201, INGN:241, or INGN:251.

44. The method of claim 38, wherein said adenoviral vector comprises a heterologous coding sequence.

45. The method of claim 43, wherein said heterologous coding sequence comprises a tumor suppressor, an inducer of apoptosis, a cell cycle regulator, a toxin, an enzyme, a hormone, a cytokine, an antisense DNA or RNA directed against an oncogene, an siRNA, a single-chain antibody, an inhibitor of angiogenesis, a metalloprotease inhibitor or a peptide hormone.

46. The method of claim 45, wherein said tumor suppressor is p53, mda-7, PTEN Rb, or p16.

47. The method of claim 45, wherein said inducer of apoptosis is Bax, Bad, Bik, Bid, or Bcl-Xₛ.

48. The method of claim 45, wherein said cell cycle regulator is p300/CBP.

49. The method of claim 45, wherein said toxin is pertussis toxin or ricin.

50. The method of claim 45, wherein said enzyme is HSV-tk.

51. The method of claim 45, wherein said hormone is LHRH analog, estrogen, progestin or an anti-androgen.

52. The method of claim 45, wherein said antisense DNA or RNA is directed to ras, raf, myb, myc, or src.

53. The method of claim 45, wherein said cytokine is tumor necrosis factor alpha, Fas ligand, tumor necrosis factor-related apoptosis-inducing ligand (TRAIL),IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-24, interferon-α, interferon-β, and interferon-γ.

54. The method of claim 45, wherein said siRNA is directed to ras, raf myb, myc, or src.

55. The method of claim 45, wherein said single-chain antibody is directed to ras, raf myb, myc, or src.

56. The method of claim 38, wherein said adenoviral vector lacks one or more regions selected from E1, E2, E3, E4, protein IX, protein IVₐ₂, and major late transcription units.

57. The method of claim 38, wherein said cotton rat cancer cell is comprised within a solid tumor.

58. The method of claim 38, wherein said cotton rat cancer cell is comprised within a metastatic lesion.

59. The method of claim 38, wherein said cotton rat cancer cell is a sarcoma cell.

60. The method of claim 38, wherein administering comprises intratumoral injection.

61. The method of claim 38, wherein admininstering comprises local, regional or systemic administration.

62. The method of claim 38, wherein administering comprises subcutaneous, intranasal, intramuscular, intravenous, intra-arterial, intraperitoneal or oral administration.

63. The method of claim 57, wherein assessing comprises measuring a change in tumor volume or diameter following treatment with said adenoviral vector.

64. The method of claim 58, wherein assessing comprises measuring metastatic growth following treatment with said adenoviral vector.

65. The method of claim 38, wherein assessing comprises measuring development of metastases following treatment with said adenoviral vector.

66. The method of claim 38, wherein assessing comprises measuring tumor cell apoptosis following treatment with said adenoviral vector.

67. The method of claim 57, wherein assessing comprises measuring tumor necrosis following treatment with said adenoviral vector.

68. The method of claim 38, wherein assessing comprises measuring tumor infiltration of adjacent tissues following treatment with said adenoviral vector.

69. The method of claim 38, wherein assessing comprises measuring production of a tumor derived compound following treatment with said adenoviral vector.

70. The method of claim 38, wherein assessing comprises measuring adenoviral-based death of said cotton rat.

71. The method of claim 38, wherein assessing comprises measuring adenoviral-based toxicity in said cotton rat.

72. The method of claim 38, wherein said adenoviral vector is administered more than once.

73. The method of claim 72, wherein assessing is performed once at the conclusion of all administrations.

74. The method of claim 72, wherein assessing is performed between at least a first and a second administration.

75. The method of claim 38, further comprising administering a second, non-adenoviral therapy to said cotton rat, and assessing the combined effect of said adenoviral vector and said second non-adenoviral therapy.
